(19) United States
(12) Patent Application Publication
(10) Pub. No.: US 2019/0321402 A1
(43) Pub. Date: Oct. 24, 2019

(54) NK CELLS FOR USE WITH ANITBODIES IN CANCER THERAPY

(71) Applicant: ONKIMMUNE LIMITED, Co. Galway (IE)

(72) Inventors: Michael Eamon Peter O'DWYER, Galway (IE); Subhashis SARKAR, Galway (IE)

(73) Assignee: ONKIMMUNE LIMITED, Co. Galway (IE)

(21) Appl. No.: 16/376,446

(22) Filed: Apr. 5, 2019

(30) Foreign Application Priority Data
Apr. 19, 2018 (EP) 18168350.9
Apr. 20, 2018 (GB) GB1806463.4

(51) Int. Cl. A61K 35/17; C07K 14/735; C07K 16/28; A61K 39/395; A61P 35/00

(52) U.S. Cl. CPC A61K 35/17; C07K 14/70535; A61K 2035/124; A61K 39/3955; A61P 35/00; C07K 16/2896

(57) ABSTRACT

Natural Killer (NK) cells and NK cell lines are modified to increase cytotoxicity, wherein the cells and compositions thereof have a use in the treatment of cancer. Modified NK cells and NK cell lines are produced via genetic modification of CD38^{low} NK cells to transiently express the Fc receptor CD16 (F158V) from mRNA introduced into the NK cell, rather than from a chromosomal coding sequence. The cytotoxicity of the modified NK cells against CD38-expressing cancer cells is increased by administration of these modified cells in combination with a CD38-binding antibody. Separately, cytotoxicity against breast cancer is exhibited by the modified NK cells in combination with

Herceptin.

### FIELD OF THE DISCLOSURE

The present disclosure relates to the modification of natural killer (NK) cells and NK cell lines to produce derivatives thereof with a more cytotoxic phenotype and improved targetting and control of this cytotoxicity. Furthermore, the present disclosure relates to methods of producing modified NK cells and NK cell lines, compositions containing the cells and cell lines and uses of said compositions in the treatment of cancer.

### BACKGROUND TO THE DISCLOSURE

Typically, immune cells require a target cell to present an antigen via the major histocompatibility complex (MHC) before triggering an immune response resulting in the death of the target cell. This allows cancer cells not presenting MHC class I to evade the majority of immune responses.

NK cells are able, however, to recognize cancer cells in the absence of MHC class I expression. Hence they perform a critical role in the body's defence against cancer.

In contrast however, under certain circumstances, cancer cells demonstrate an ability to dampen the cytotoxic activity of NK cells, through expression of ligands that bind inhibitory receptors on the NK cell membrane. Resistance to cancer can involve a balance between these and other factors.

Cytotoxicity, in this context, indicates the ability of immune effector cells, e.g. NK cells, to induce cancer cell death, e.g. by releasing cytolytic compounds or by binding receptors on cancer cell membranes and inducing apoptosis of said cancer cells. Cytotoxicity is affected not only by signals that induce release of cytolytic compounds but also by signals that inhibit their release. An increase in cytotoxicity will therefore lead to more efficient killing of cancer cells, with less chance of the cancer cell dampening the cytotoxic activity of the NK, as mentioned above.

Despite significant investment in a variety of physical, pharmaceutical and other therapies, human cancer remains a significant cause of mortality across all age groups. NK cells are cytotoxic lymphocytes, with distinct phenotypes and effector functions that differ from e.g. natural killer T (NK-T) cells. For example, while NK-T cells express both CD3 and T cell antigen receptors (TCRs), NK cells do not. NK cells are generally found to express the markers CD16 and CD56, wherein CD16 functions as an Fc receptor and mediates antibody dependent cell-mediated cytotoxicity (ADCC) which can be assayed using methods known in the art (Alpert et al. 2012) and is discussed further below. KHYG-1 (see below) is a notable exception in this regard. Despite NK cells being naturally cytotoxic, NK cell lines with increased cytotoxicity have been developed. NK-92 and KHYG-1 represent two NK cell lines that have been researched extensively and show promise in cancer therapeutics (Swift et al. 2011; Swift et al. 2012). Due to the low expression levels of CD16 on KHYG-1 cells, it has previously been considered worthwhile to knock-in CD16, so as to produce modified KHYG-1 cells that stably express CD16 (Kobayashi et al. 2014). Stable transfection of NK cells with a high affinity CD16 variant (F158V) has also been carried out (WO 2016/077734; U.S. Patent Appl. Publication No. 2018/0325951, which is incorporated by reference in its entirety).

A problem with both endogenous expression of Fc receptors and expression of Fc receptors following genetic knock-in in NK cells is that during therapy in the presence of target cells (i.e. during NK cell activation) the Fc receptors are susceptible to quick cleavage at the cell membrane, e.g. by metalloproteases. Replacement of the cleaved CD16 is slow and leads to the NK cells having reduced cytotoxicity as a result of a decreased ability to participate in ADCC (Harrison et al. 1991; Tosi et al. 1992; Wang et al. 2013; Romee et al. 2013). This problem has led to the development of a non-cleavable version of Fc receptor CD16 (Jing et al. 2015).

Adoptive cellular immunotherapy for use in cancer treatment commonly involves administration of natural and modified T cells to a patient. T cells can be modified in various ways, e.g. genetically, so as to express receptors and/or ligands that bind specifically to certain target cancer cells. Transfection of T cells with high-affinity T cell receptors (TCRs) and chimeric antigen receptors (CARs), specific for cancer cell antigens, can give rise to highly reactive cancer-specific T cell responses. A major limitation of this immunotherapeutic approach is that T cells must either be obtained from the patient for autologous ex vivo expansion or MHC-matched T cells must be used to avoid immunological eradication immediately following transfer of the cells to the patient or, in some cases, the onset of graft-vs-host disease (GVHD). Additionally, successfully transferred T cells often survive for prolonged periods of time in the circulation, making it difficult to control persistent side-effects resulting from treatment.

In haplotype transplantation, the graft-versus-leukaemia effect is believed to be mediated by NK cells when there is a KIR inhibitory receptor-ligand mismatch, which can lead to improved survival in the treatment of AML (Ruggeri, Capanni et al. 2002; Ruggeri, Mancusi et al. 2005). Furthermore, rapid NK recovery is associated with better outcome and a stronger graft-vs-leukaemia (GVL) effect in patients undergoing haplotype T-depleted hematopoietic cell transplantation (HCT) in AML (Savani, Mielke et al. 2007). Other trials have used haploidentical NK cells expanded ex vivo to treat AML in adults (Miller, Soignier et al. 2005) and children (Rubnitz, Inaba et al. 2010).

Several permanent NK cell lines have been established, and the most notable is NK-92, derived from a patient with non-Hodgkin's lymphoma expressing typical NK cell markers, with the exception of CD16 (Fc gamma receptor III). NK-92 has undergone extensive preclinical testing and exhibits superior lysis against a broad range of tumours compared with activated NK cells and lymphokine-activated killer (LAK) cells (Gong, Maki et al. 1994). Cytotoxicity of NK-92 cells against primary AML has been established (Yan, Steinherz et al. 1998). Another NK cell line, KHYG-1, has been identified as a potential contender for clinical use (Suck et al. 2005) but is reported by some as having reduced cytotoxicity so has received less attention than NK-92.

The identities of specific cancer markers are also sought to help in the fight against cancer. However, a problem with many known cancer markers is that they are also expressed, perhaps at different levels, on healthy cells, meaning that ‘targeted’ therapies will nevertheless inevitably result in a certain amount of self-targetting. One example of such a cell surface marker is CD38, also known as cyclic ADP ribose hydrolase, which is a glycoprotein found on the surface of many immune cells (white blood cells), including CD4⁺, CD8⁺, B lymphocytes and natural killer cells. The CD38 protein is a marker of cell activation and is known to be present on certain cancer cells. It has been connected to leukaemias, myelomas such as multiple myeloma, and solid tumours. CD38-expressing cancers have been targeted with anti-CD38 antibodies such as Daratumumab (an anti-CD38 IgG1k monoclonal antibody). However, administration of Daratumumab has been reported to cause rapid depletion of CD38-expressing NK cells in patients (Casneuf T, et al. Blood Advances. 2017).

Thus, there exists a need for alternative and preferably improved cell based therapies with greater selectivity for cancer cells.

An object of the present disclosure is to address one or more problems identified above, e.g. to provide NK cells and NK cell lines that target cancer cells with high selectivity and preferably have a more cytotoxic phenotype. A further object is to provide methods for producing modified NK cells and NK cell lines, compositions containing the cells or cell lines and uses of such in the treatment of cancers. More particular embodiments aim to provide treatments for identified cancers, e.g. blood cancers, including leukaemia. Specific embodiments aim at combining two or more modifications of NK cells and NK cell lines to further enhance the cytotoxicity of the modified cells.

### Summary of the Disclosure

There are provided herein modified NK cells and NK cell lines with a more cytotoxic phenotype, and methods of making the cells and cell lines. Also provided are compositions of modified NK cells and NK cell lines, and uses of said compositions for treating cancer. Cytotoxicity in this context, as above, indicates killing of tumour cells, especially cancer cells.

Accordingly, the present disclosure provides a natural killer (NK) cell or NK cell line that has been genetically modified to increase its cytotoxicity.

As described in detail below in examples, NK cells and NK cell lines have been modified so as to increase their cytotoxic activity against cancer.

Together, the NK cells and NK cell lines of the disclosure will also be indicated as the NK cells (unless the context requires otherwise).

Accordingly, the disclosure provides a natural killer (NK) cell expressing an Fc receptor. In some embodiments, the NK cell is CD38^{low}.

CD38^{low} is a term indicating low to no expression of CD38 on the cell surface. The definition of low CD38 expression is normally determined experimentally in relation to populations of cells resolved using FACS that show lower or no CD38 expression. In some embodiments, this determination is made with reference to a separately resolved population of cells showing higher or positive CD38 expression.

The level of CD38 present on the surface of an NK cell may be conveniently quantified with reference to well-characterised cells or cell lines that are known to express CD38. Such quantification may be carried out by FACS with an anti-CD38 antibody. Such cells or cell lines include the myeloma cell lines RPMI 8226, MM.1S and H929, and primary expanded NK cells and NK cell line NK-92. The level of CD38 expression defined as CD38^{low} is defined as being <35% of the mean fluorescence intensity (MFI) signal obtained from anti-CD38 FACS of RPMI 8226 cells. In some embodiments, the level of CD38 expression is equal to or less than 30% of the mean fluorescence intensity (MFI) signal obtained from anti-CD38 FACS of RPMI 8226 cells. In some embodiments, the level of CD38 expression is less than or equal to 25% of the mean fluorescence intensity (MFI) signal obtained from anti-CD38 FACS of RPMI 8226 cells. In some embodiments, the level of CD38 expression is equal to or less than 20% of the mean fluorescence intensity (MFI) signal obtained from anti-CD38 FACS of RPMI 8226 cells. In some embodiments, the level of CD38 expression is equal to or less than 15% of the mean fluorescence intensity (MFI) signal obtained from anti-CD38 FACS of RPMI 8226 cells. In some embodiments, CD38 expression is equal to or less than 10% of the MFI obtained from other NK cells (primary expanded NK cells and NK-92). In some embodiments, the level of CD38 expression is equal to or less than 5% of the mean fluorescence intensity (MFI) signal obtained from anti-CD38 FACS of RPMI 8226 cells.

As noted above, administration of an anti-CD38 antibody has been reported to cause rapid depletion of CD38-expressing NK cells in patients. Use of a CD38^{low} NK cell line is advantageous as CD38^{low} NK cells can be used in combination with an anti-CD38 antibody without the administered NK cells of the disclosure being targeted against themselves. Thus use of CD38^{low} NK cells yields better and more effective killing of CD38-expressing cells, including cancer cells, as the potency of the NK cells of the disclosure is better preserved/maintained by not being degraded by self-targetting.

The Fc receptor on NK cells of the disclosure recognizes IgG and may be CD16 or FcγRIII. Other Fc receptors may also be used, such as CD32 and CD64. The Fc receptor on the NK cell in examples below is CD16; all illustrate the disclosure. Activation of CD16 by IgG causes the release of cytotoxic mediators like perforin and granzyme that enter the target cell and promote cell death by triggering apoptosis. This process is known as antibody-dependent cell-mediated cytotoxicity (ADCC).

15% of the population expresses a higher affinity form of CD16, due to a single point polymorphism (F158V) and this has been linked to higher responsiveness to therapeutic monoclonal antibodies. This variant is known as high-affinity CD16 or (HA CD16). Accordingly, in some embodiments the CD16 receptor comprises the amino acid substitution mutation F158V.

The Fc receptor may be expressed from an extra-chromosomal nucleic acid. Hence, the nucleic acid is exogenous and is introduced into the NK cell, not being located on the NK cell chromosome. In some embodiments, the Fc receptor is expressed at levels greater than found on wild type cells, i.e. the Fc receptor is over expressed, e.g. at a level 10% or higher than on wild type cells In some embodiments, the Fc receptor is expressed at a level 20% or higher than on wild type cells. In some embodiments, the Fc receptor is expressed at a level 30% or higher than on wild type cells. Over expression of the Fc receptor has the advantage of increasing the stimulus to the NK cell triggered by the receptor binding an antibody and therefore the cytotoxic, and therapeutic, effect of the NK cells of the disclosure.

The extra-chromosomal nucleic acid may be RNA. In some embodiments, the extra-chromosomal nucleic acid is mRNA. However, the extra-chromosomal nucleic acid may be, or be located on, a nucleic acid vector. A variety of vectors may be employed including: DNA vectors, plasmids and viral vectors (DNA or RNA). In some embodiments, the vector does not replicate in NK cells.

Expression of the Fc receptor may be transient. That is the Fc receptor is only expressed for a limited period of time. An advantage of this transient expression of the Fc receptor is that the potent cytotoxicity of the NK cells of the disclosure can be better controlled in order to avoid, ameliorate and/or minimise off-target/side effects caused by NK cells interacting with, for example, non-cancerous CD38-expressing cells. Thus transient expression allows the delivery of controlled ‘pulses’ of NK cytotoxic activity.

Control of expression in order to yield, transient expression can be achieved by a number of methods. One uses transfected mRNA encoding the Fc receptor, which will naturally be degraded in the cell over time and is not capable of replication. Accordingly, the amount of Fc receptor produced from such transfected mRNA will also reduce over time. Alternatively, other nucleic acid vectors, including inducible vectors and/or vectors capable of replication can be used to express the Fc receptor in a controlled manner.

Expression of the Fc receptor may be maintained for (meaning for at least) 600 hours (25 days), 480 hours, (20 days), 360 hours, (15 days), 240 hours (10 days), 120 hours (5 days), 96 hours (4 days), 72 hours (3 days), 48-hours (2 days) or 24 hours (1 day). In some embodiments, expression of the Fc receptor may be maintained for (meaning for at least) 600 hours (25 days). In some embodiments, expression of the Fc receptor may be maintained for (meaning for at least) 480 hours, (20 days). In some embodiments, expression of the Fc receptor may be maintained for (meaning for at least) 360 hours, (15 days). In some embodiments, expression of the Fc receptor may be maintained for (meaning for at least) 240 hours (10 days). In some embodiments, expression of the Fc receptor may be maintained for (meaning for at least) 120 hours (5 days). In some embodiments, expression of the Fc receptor may be maintained for (meaning for at least) 96 hours (4 days). In some embodiments, expression of the Fc receptor may be maintained for (meaning for at least) 72 hours (3 days). In some embodiments, expression of the Fc receptor may be maintained for (meaning for at least) 48-hours (2 days). In some embodiments, expression of the Fc receptor may be maintained for (meaning for at least) 24 hours (1 day). In particular embodiments, expression of the Fc receptor is maintained for 120 hours. The expression of the extra-chromosomal nucleic acid Fc receptor may be controlled and/or modulated by the amount and transcriptional activity of the extra-chromosomal nucleic acid used.

NK cells of the disclosure are especially suited for use with an antibody via a combination therapy; one such antibody, used in examples herein, binds CD38. Another antibody, used in a separate example, binds a breast cancer marker. Hence, the antibody suitably binds a cancer cell. The CD38-binding antibody may be a monoclonal antibody; in some embodiments, the antibody is Daratumumab, an anti-CD38 IgG1κ monoclonal antibody though other CD38—targeting antibodies are known and suitable. Accordingly, an NK cell as described herein may be used with and potentiate the therapeutic activity of Daratumumab against cancer cells. In some embodiments, this therapeutic activity is against multiple myeloma cell lines and/or primary myeloma cells. In some embodiments, the NK cell is of the KHYG1 cell line (which is CD38^{low}) or a derivative thereof. Unexpectedly, KHYG-1 cells are found to have much lower levels of CD38 expression compared to either primary peripheral blood derived expanded NK cells or NK-92. This has the advantage of being able to provide a combination therapy of CD16-expressing KHYG-1 with Daratumumab (a CD38 monoclonal antibody) without significant killing of the KHYG-1 cells (NK induced fratricide). This is not the case with, for example, cells of line NK-92 and primary NK cells where a Daratumumab combination therapy yields significant NK killing; such ‘self-targetting’ yielding collateral damage to the therapeutic population of NK cells is clearly disadvantageous.

Furthermore, surprisingly, KHYG-1 cells have much lower levels of CD16 expression compared to normal expanded NK cells. This is advantageous as it allows for the level and timing of CD16 activity, and hence the targeted cytotoxicity yielded by binding of an antibody to CD16, to be modulated via control of expression of CD16 as per the transient expression embodiments of the disclosure.

Use of the KHYG-1 cell line is also advantageous because expression of introduced CD16 as a cell surface protein receptor has been found to be very stable in this cell line. That is, the level of CD16 did not decline significantly on co-culture with target tumour cells. This process of CD16 loss from the cell surface following co-culture with tumour cells is known as shedding. Typically, on activation of NK cells CD16 is shed following activation of a metalloprotease enzyme, which cleaves CD16 at a specific cleavage site. Consequently, NK cells typically quickly lose a large proportion of their CD16 receptors and thus become less effective at binding antibodies. That NK cells, especially KHYG-1 cells, modified in accordance with the disclosure, are not or much less susceptible to this shedding of CD16 was an unexpected finding that markedly contrasts with the shedding characteristics of other known NK cells and NK cell lines.

The disclosure also provides an NK cell as described herein for use in treating cancer. In some embodiments, the NK cell is used in combination with an anti-cancer antibody.

Suitable antibodies include, but are not limited to, Daratumumab, Trastuzumab (Herceptin), Alemtuzumab, Brentuximab, Blinatumomab, Pankomab, Avelumab, Durvalumab and Atezolizumab. In some embodiments, the antibody is Daratumumab (Darzalex). In some embodiments, the antibody is Trastuzumab (Herceptin).

Diseases particularly treatable according to the disclosure include cancers, solid cancers and blood cancers, more particularly breast cancers, ovarian cancers, colorectal cancers, lymphomas, myelomas, multiple myelomas, leukemias and specifically acute myeloid leukaemia. Tumours and cancers in humans in particular can be treated. References to tumours herein include references to neoplasms.

In particular embodiments, the cancer treated using an NK of the disclosure is a blood cancer. In some embodiments, the cancer is multiple myeloma, acute myeloid leukemia or any other CD38 antigen expressing hematological cancer, in particular when the NK cell is in combination with an antibody that binds CD38. In other particular embodiments, the cancer is a solid cancer. In some embodiments, the cancer is breast cancer.

The disclosure further provides a pharmaceutical composition comprising an NK cell as described herein and an antibody. Accordingly, the pharmaceutical composition may comprise a CD38-binding antibody and be for use in treating a CD38-expressing cancer.

The disclosure further provides a pharmaceutical kit comprising an NK cell as described herein and an antibody. In some embodiments, the pharmaceutical kit further comprises instructions for administration of the NK cell and the antibody to a patient. In some embodiments, the pharmaceutical kit further comprises instructions for administration of the NK cell or the antibody to a patient. In some embodiments, the administration comprises treatment with an NK cell and with an antibody. In some embodiments, the pharmaceutical kit further comprises a notice in the form prescribed by a governmental agency regulating the manufacture, use or sale of pharmaceuticals or biological products, which notice reflects approval by the agency of manufacture, use or sale for human administration. In some embodiments, the kit comprises an antibody that binds CD38. In some embodiments, the antibody binds HER2. In some embodiments, the antibody is selected from the group consisting of Daratumumab, Trastuzumab, Alemtuzumab, Brentuximab, Blinatumomab, Pankomab, Avelumab, Durvalumab and Atezolizumab.

The disclosure also provides a method of preparing a NK cell expressing an Fc receptor comprising the step of: transfecting a NK cell to introduce a nucleic acid expressing the Fc receptor into the cell. Transfection may be by nucleofection or electroporation. In some embodiments, transfection is by electroporation. In some embodiments, transfection results in transient expression of the Fc receptor. In some embodiments, the NK cell is CD38^{low}.

Utilization of clinical grade electroporation system (e.g. the Maxcyte GT system, which has provided viability and receptor expression>80%) provides scalability for clinical use of methods of the disclosure. The use of electroporation has the advantage of providing a mRNA based therapeutic treatment strategy which can be combined e.g. with Daratumumab, or another suitable antibody which binds to CD38, that is safer in that the period of expression of the receptor activating the cytotoxic activity of the NK cells of the disclosure is, by default, limited to the lifetime of the mRNA electroporated into the cells. Accordingly, electroporation has the advantage of providing a “safer” mRNA-based “off-the-shelf” therapeutic treatment strategy that can be combined with Daratumumab, or another suitable antibody which binds to CD38.

In addition to the mRNA based therapeutic treatment strategies being limited to the lifetime of the mRNA electroporated into the cells, the resulting proteins are found to be formed and sent to the cell membrane in a continuous manner. In other words, mRNA based therapeutic treatment strategies provide the advantage that the resulting protein function is not easily disabled by e.g. external factors. It is known, for example, that endogenous CD16 is cleaved by metalloproteases in vivo which can limit ADCC and hence cancer cell killing. In the art, non-cleavable versions of CD16 have been tried. In the disclosure, the constant trafficking of CD16 expressed via an mRNA based therapeutic treatment strategy (e.g. Maxcyte GT system) to the NK cell membrane overcomes the problems associated with endogenous CD16 cleavage, thereby providing an NK cell with an enhanced ability to kill cancer cells via ADCC.

The disclosure also provides a method for treating cancer comprising administering an NK cell of the disclosure to a patient suffering from cancer. In some embodiments, the administration is in combination with an antibody that binds to the Fc receptor of the NK cell. In some embodiments, this antibody binds CD38. In some embodiments, the antibody is Daratumumab.

An advantage of the disclosure is that the NK cells and lines are targeted to cancer. Whether used alone or in combination with other therapeutic components or as cells/lines with other modifications the cells can be used in cancer therapy.

In some embodiments, the present disclosure provides using NK cells to allow for the risk of such self-targetting and provide further elements to overcome this.

Despite the risk of self targetting effects, e.g. that CD38 is expressed on other cells within the patient so that one or more of these cell types can be destroyed by the therapeutic NK cells of the disclosure, the therapies of the disclosure may be carried out with reduced durations of exposure to the therapeutic NK cells, mitigating the risks. NK cells generally do not survive for long periods in circulation, perhaps up to several weeks (though this varies), reducing the self-targetting risk. The present disclosure uses NK cells, not T cells which persist in the patient for months and even many months, hence the therapeutic index for the NK cells of the disclosure is expected to be much wider. Thus, an acceptable therapeutic effect can be achieved without significant negative side effects.

NK cells or cell lines according to the disclosure may also be treated or pre-treated to render them incapable of division. This results in further reduced lifetime in circulation in the patient, e.g. in comparison with T cells, further mitigating the risks above, and also with reduced or absent propensity to form tumours in a patient. These features are described also in more detail below.

NK cells and cell lines of the provided disclosure are for use in treating cancer in a patient, especially a human. The cancer is suitably a solid cancer, e.g. breast cancer, ovarian cancer or colorectal cancer. It may be a blood cancer, especially a blood cancer selected from the group consisting of acute lymphocytic leukaemia (ALL), acute myeloid leukaemia (AML), chronic lymphocytic leukaemia (CLL), chronic myeloid leukaemia (CML), Hodgkin's lymphoma, non-Hodgkin's lymphoma, including T-cell lymphomas and B-cell lymphomas, asymptomatic myeloma, smoldering multiple myeloma (SMM), active myeloma and light chain myeloma; in particular it is a leukaemia or multiple myeloma.

As is well known in the art, immunosuppressive factors (e.g. TGF-β) act as important promoters of malignant cell growth (De Visser et al. 1999). One mechanism is by deactivating NK cell responses, i.e. reducing NK cell cytotoxicity. As shown in specific examples below, transient CD16 expression on NK cells according to the disclosure produced NK cells that were less susceptible to the cytotoxicity-dampening effects of immunosuppressive factors.

As such, surprisingly but advantageously, the disclosure provides NK cells that are less susceptible to immunosuppression. The disclosure thus further provides the NK cells, compositions thereof, uses thereof, medical uses thereof and methods of treatment using the cells, whereby cancer is treated with reduced or substantially absent downregulation of cytotoxicity by one or more immunosuppressive factors. A prejudice with respect to in vivo cancer therapies is the known phenomenon of immunosuppression; this may steer a skilled person away from in vivo use of cell therapies which at first sight look promising in vitro. TGF-β, lactate and PGE₂ are each known to be a feature of the cancer microenvironment and immunosuppressive. Data herein have demonstrated maintenance of a cytotoxic NK phenotype even in the presence of these factors.

In preparing NK cells, a genetic modification may occur before the cell has differentiated into an NK cell. For example, pluripotent stem cells (e.g. iPSCs) can be genetically modified then differentiated to produce genetically modified CAR NK cells with increased cytotoxicity.

In certain embodiments of the disclosure NK cells are provided that are further modified so as to have reduced or absent checkpoint inhibitory receptor function. NK cells may be produced that have one or more checkpoint inhibitory receptor genes knocked out, mutated or absent. In some embodiments, these receptors are specific checkpoint inhibitory receptors. In some embodiments, this checkpoint inhibitory receptor is Killer-cell Immunoglobulin-like Receptor (KIR), a receptor for MHC class I molecules on NK cells. In other embodiments, NK cells are provided in which one or more inhibitory receptor signalling pathways are knocked out or exhibit reduced function—the result again being reduced or absent inhibitory receptor function.

As used herein, references to inhibitory receptors generally indicate a receptor expressed on the plasma membrane of an immune effector cell, e.g. a NK cell, whereupon binding its complementary ligand resulting intracellular signals are responsible for reducing the cytotoxicity of said immune effector cell. These inhibitory receptors are expressed during both ‘resting’ and ‘activated’ states of the immune effector cell and are often associated with providing the immune system with a ‘self-tolerance’ mechanism that inhibits cytotoxic responses against cells and tissues of the body. An example is the inhibitory receptor family ‘KIR’ which are expressed on NK cells and recognize MHC class I expressed on healthy cells of the body.

It is preferred to reduce function of checkpoint inhibitory receptors over other inhibitory receptors, due to the expression of the former following NK cell activation. The normal or ‘classical’ inhibitory receptors, such as the majority of the KIR family, NKG2A and LIR-2, bind MHC class I and are therefore primarily involved in reducing the problem of self-targetting. In some embodiments, therefore, checkpoint inhibitory receptors are knocked out. Reduced or absent function of these receptors according to the disclosure prevents cancer cells from suppressing immune effector function (which can otherwise occur if the receptors were fully functional). Thus a key advantage of these embodiments of the disclosure lies in NK cells that are less susceptible to suppression of their cytotoxic activities by cancer cells; as a result they are useful in cancer treatment.

Lacking a gene can indicate either a full or partial deletion, mutation or otherwise that results in no functional gene product being expressed. In embodiments, the NK cell lacks the gene encoding wildtype CD16. NK cells of the disclosure may thus lack wildtype CD16 and express high affinity Fc receptor, e.g. high affinity CD16 extrachromosomally, such as via mRNA as described herein. In further embodiments the NK cell lacks genes encoding the members of the KIR family. In yet further embodiments, the NK cell does not express CD16 from the gene encoding CD16. In yet further embodiments the NK cell does not express the members of the KIR family from the genes encoding these proteins.

In some embodiments, the present disclosure provides adapting the modified NK cells and NK cell lines to better home to specific target regions of the body. NK cells of the disclosure may be targeted to specific cancer cell locations. In preferred embodiments for treatment of blood cancers, NK effectors of the disclosure are adapted to home to bone marrow. Specific NK cells are modified by fucosylation and/or sialylation to home to bone marrow. This may be achieved by genetically modifying the NK cells to express the appropriate fucosyltransferase and/or sialyltransferase, respectively. Increased homing of NK effector cells to tumour sites may also be made possible by disruption of the tumour vasculature, e.g. by metronomic chemotherapy, or by using drugs targetting angiogenesis (Melero et al, 2014) to normalize NK cell infiltration via cancer blood vessels. Notably, the KHYG1 cell line has the particular advantage of homing to the bone marrow.

Modified NK cells, NK cell lines and compositions thereof described herein, above and below, are suitable for treatment of cancer, in particular cancer in humans, e.g. for treatment of cancers of blood cells or solid cancers. The NK cells and derivatives are preferably human NK cells. In some embodiments, for human therapy, human NK cells are used. The disclosure also provides methods of treating cancer in humans comprising administering an effective amount of the cells or lines or compositions.

Various routes of administration will be known to the skilled person to deliver active agents and combinations thereof to a patient in need. Embodiments of the disclosure are for blood cancer treatment. Administration of the modified NK cells and/or NK cell lines can be systemic or localized, such as for example intravenously or via the intraperitoneal route.

In other embodiments, active agent is administered more directly. Thus administration can be directly intratumoural, suitable especially for solid tumours.

NK cells in general are believed suitable for the methods, uses and compositions of the disclosure. As per cells used in certain examples herein, the NK cell can be a NK cell obtained from a cancer cell line. In some embodiments, Advantageously, a NK cell, treated to reduce its tumourigenicity, for example by rendering it mortal and/or incapable of dividing, can be obtained from a blood cancer cell line and used in methods of the disclosure to treat blood cancer.

To render a cancer-derived NK cell more acceptable for therapeutic use, it is generally treated or pre-treated in some way to reduce or remove its propensity to form tumours in the patient. Specific modified NK cell lines used in examples are safe because they have been rendered incapable of division; they are irradiated and retain their killing ability but die within about 3-4 days. Specific cells and cell lines are hence incapable of proliferation, e.g. as a result of irradiation. Treatments of potential NK cells for use in the methods herein include irradiation to prevent them from dividing and forming a tumour in vivo and genetic modification to reduce tumourigenicity, e.g. to insert a sequence encoding a suicide gene that can be activated to prevent the cells from dividing and forming a tumour in vivo. Suicide genes can be turned on by exogenous, e.g. circulating, agents that then cause cell death in those cells expressing the gene. A further alternative is the use of monoclonal antibodies targetting specific NK cells of the therapy. CD52, for example, is expressed on KHYG-1 cells and binding of monoclonal antibodies to this marker can result in antibody-dependent cell-mediated cytotoxicity (ADCC) and KHYG-1 cell death.

As discussed in an article published by Suck et al, 2006, cancer-derived NK cells and cell lines are easily irradiated using irradiators such as the Gammacell 3000 Elan. A source of Cesium-137 is used to control the dosing of radiation and a dose-response curve between, for example, 1 Gy and 50 Gy can be used to determine the optimal dose for eliminating the proliferative capacity of the cells, whilst maintaining the benefits of increased cytotoxicity. This is achieved by assaying the cells for cytotoxicity after each dose of radiation has been administered.

There are significant benefits of using an irradiated NK cell line for adoptive cellular immunotherapy over the well-established autologous or MHC-matched T cell approach. Firstly, the use of a NK cell line with a highly proliferative nature means expansion of modified NK cell lines can be achieved more easily and on a commercial level. Irradiation of the modified NK cell line can then be carried out prior to administration of the cells to the patient. These irradiated cells, which retain their useful cytotoxicity, have a limited life span and, unlike modified T cells, will not circulate for long periods of time causing persistent side-effects.

The present disclosure is now described in more and specific details in relation to the production of NK cell line KHYG-1 derivatives, modified to exhibit more cytotoxic activity through an ability to overexpress the Fc receptor CD16. Such cells are demonstrated to have an increased cytotoxic effect when used in combination with the anti-CD38 monoclonal antibody Daratumumab.

### EXAMPLES

The disclosure is now illustrated in specific embodiments with reference to the accompanying drawings in which:

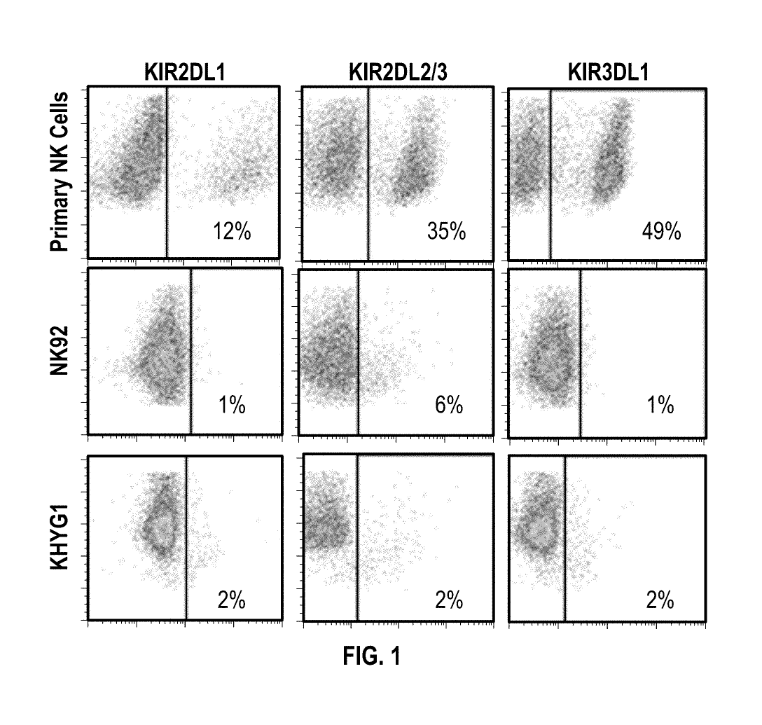

FIGS. 1A-1C show KHYG1 NK cells lack KIR inhibitory receptors on their surface.

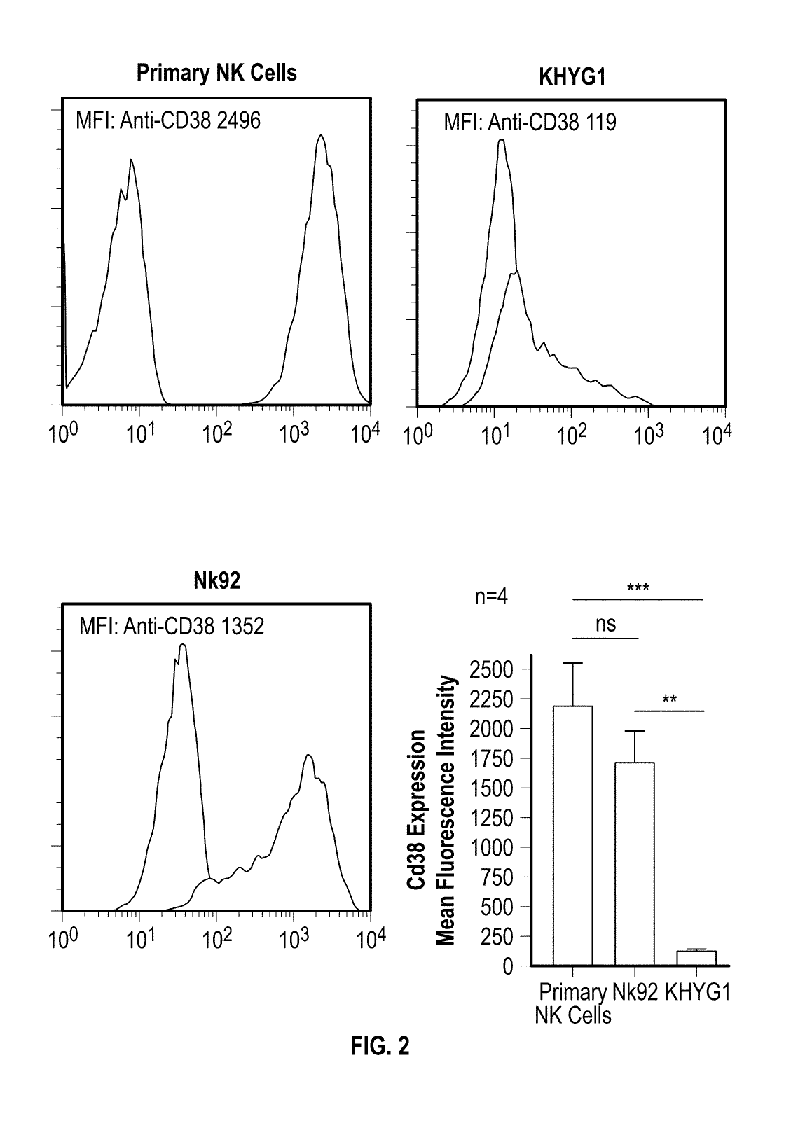

FIGS. 2A-2D show KHYG1 NK cells exhibit a low abundance of CD38 receptors on their surface (i.e. they are CD38^{low}). Sample size n=4.

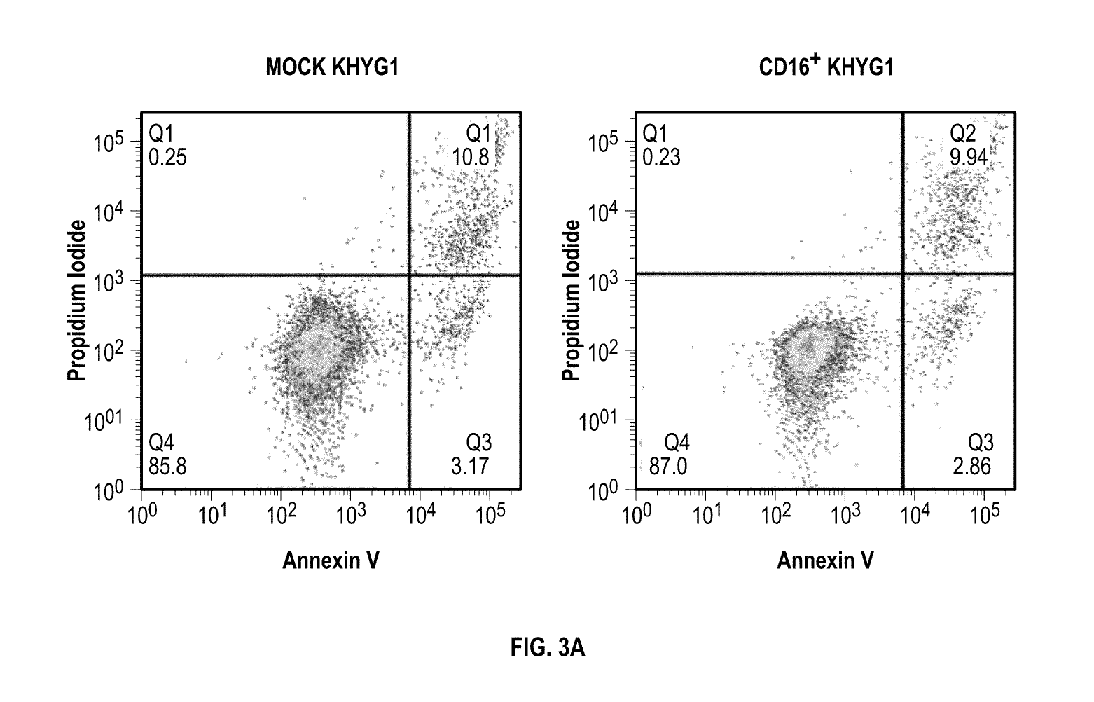

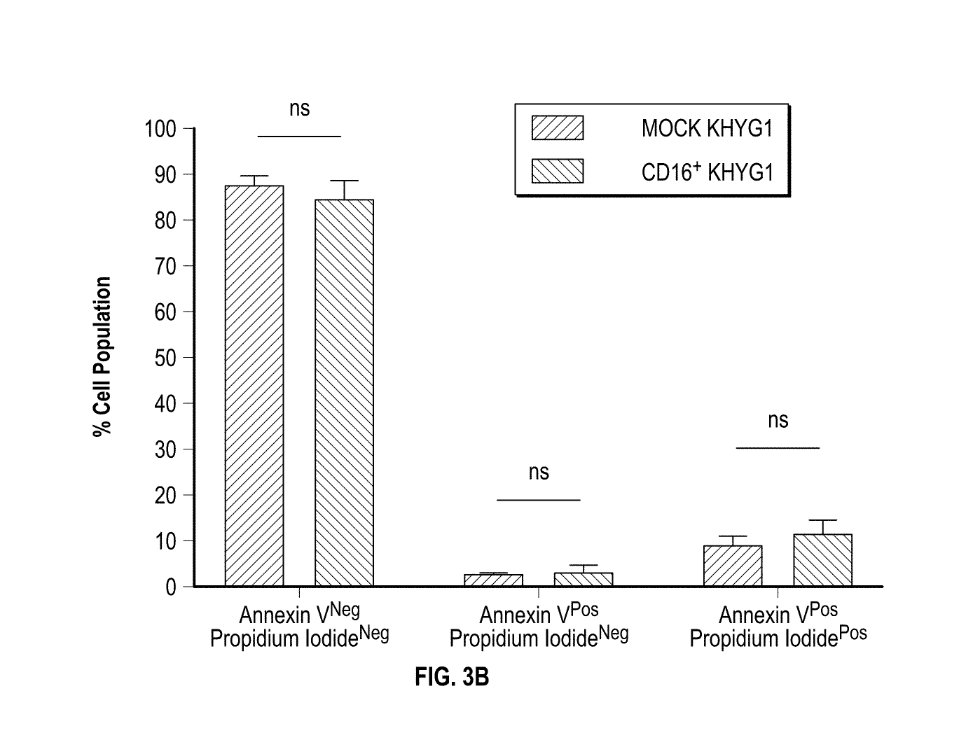

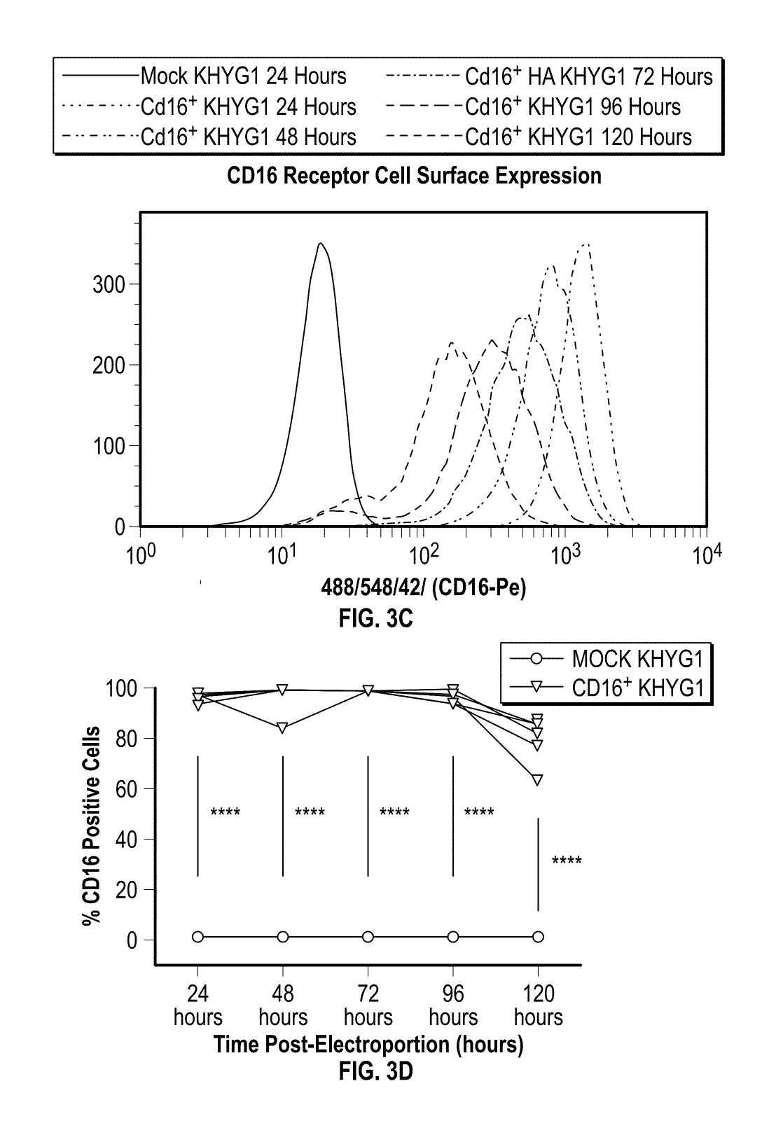

FIGS. 3A-3E show that KHYG1 cells electroporated with CD16 mRNA maintain viability and express CD16 receptors on their surface over a period of 120 hours.

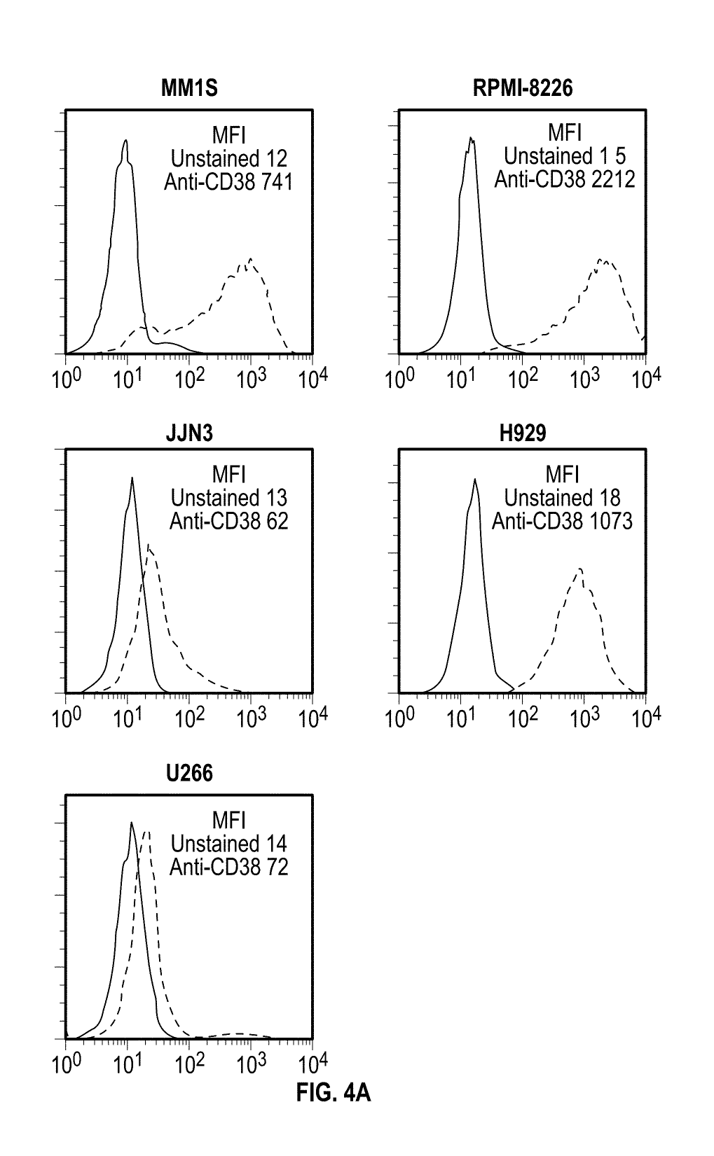

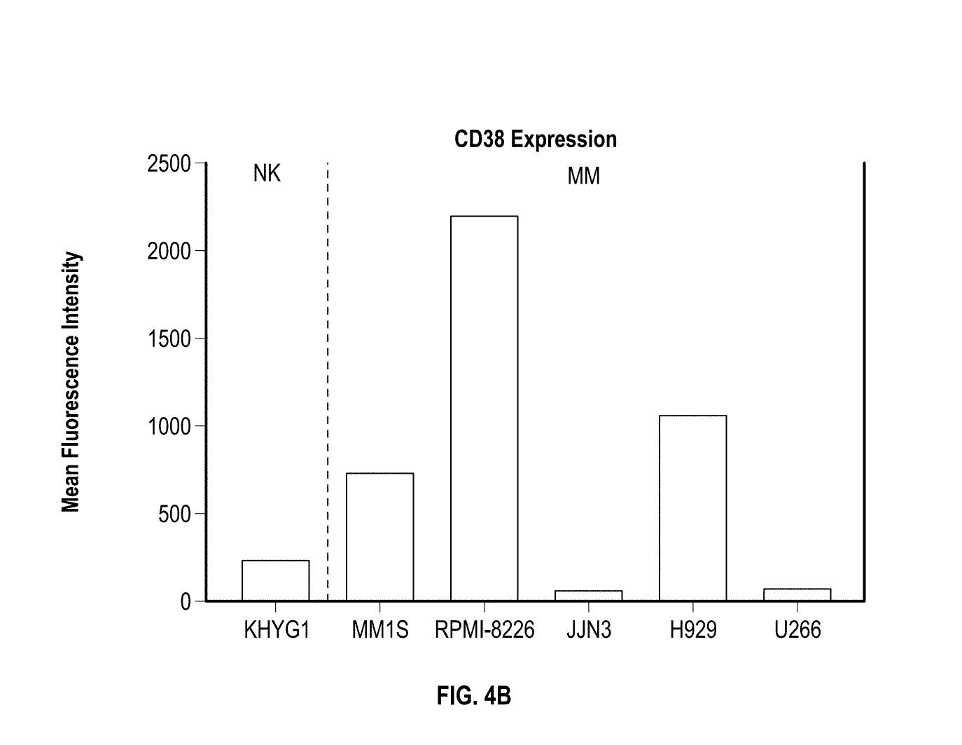

FIGS. 4A-4F show the level of CD38 expression on multiple myeloma and NK cell lines

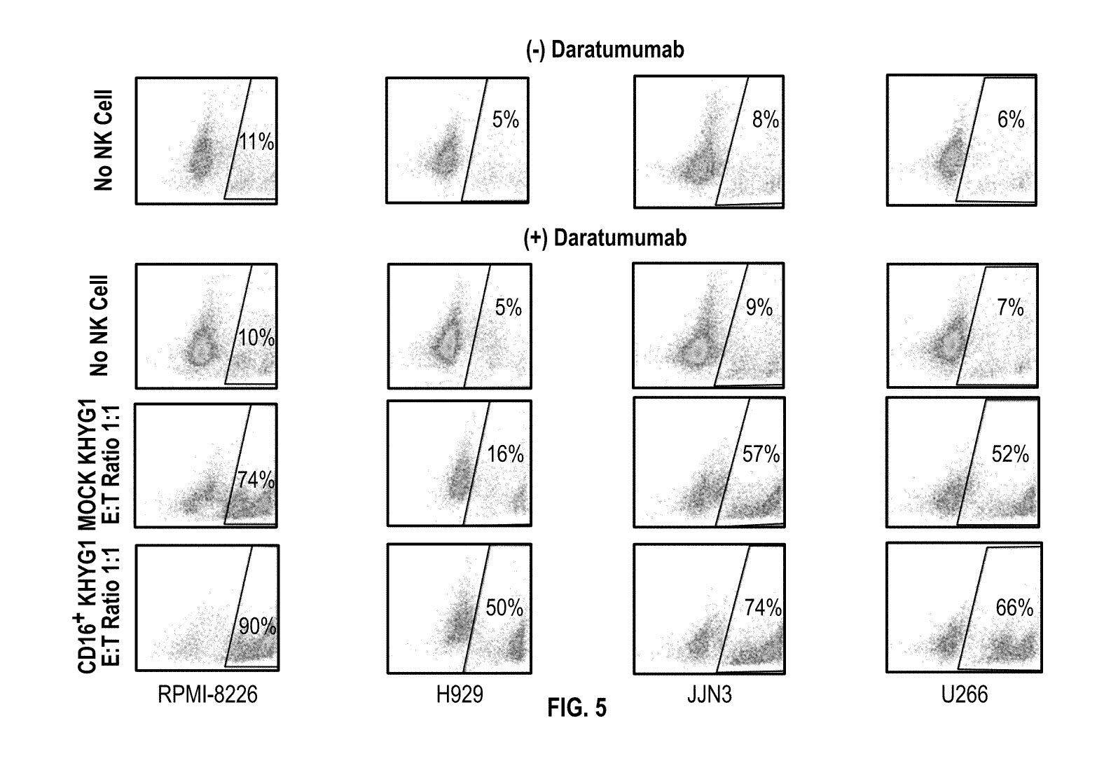

FIGS. 5A-5B show that CD16⁺ KHYG1 NK cells enhance the therapeutic activity of Daratumumab against myeloma cell lines more than mock electroporated CD16^{negative} KHYG1 NK cells.

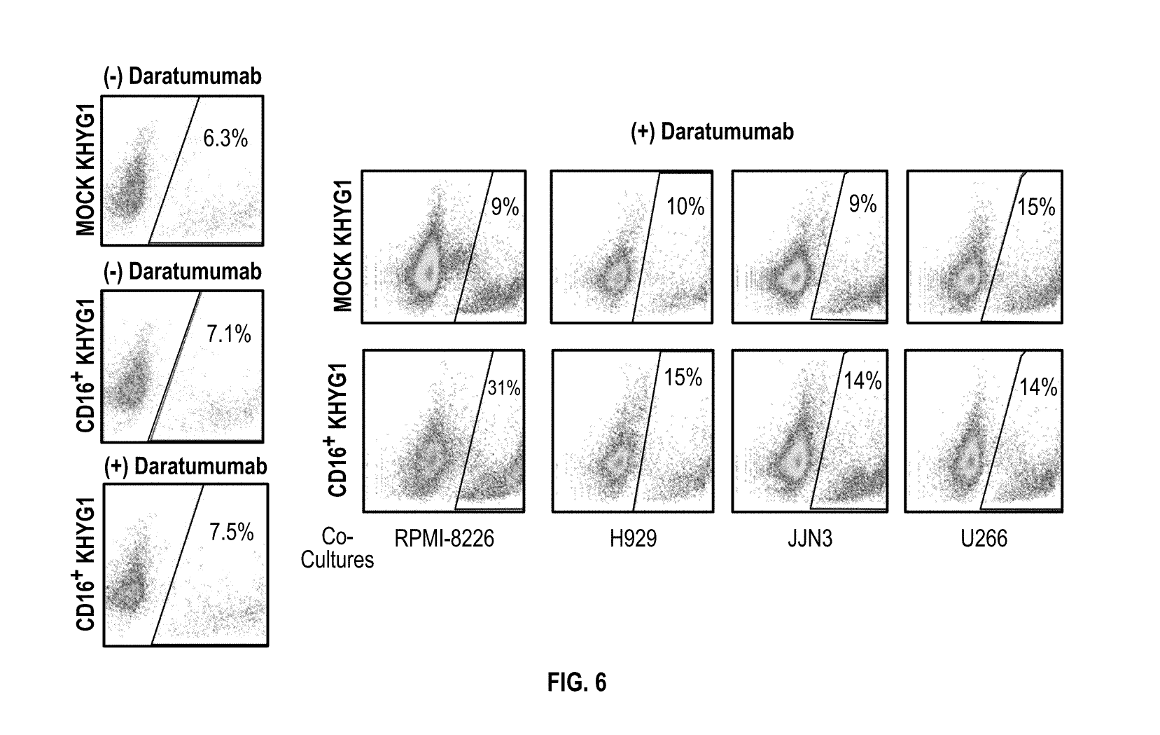

FIGS. 6A-6B show Daratumumab per se is not toxic to CD38^{low} CD16+ KHYG1 NK cells, i.e. Daratumumab treatment causes minimal collateral damage to these cells.

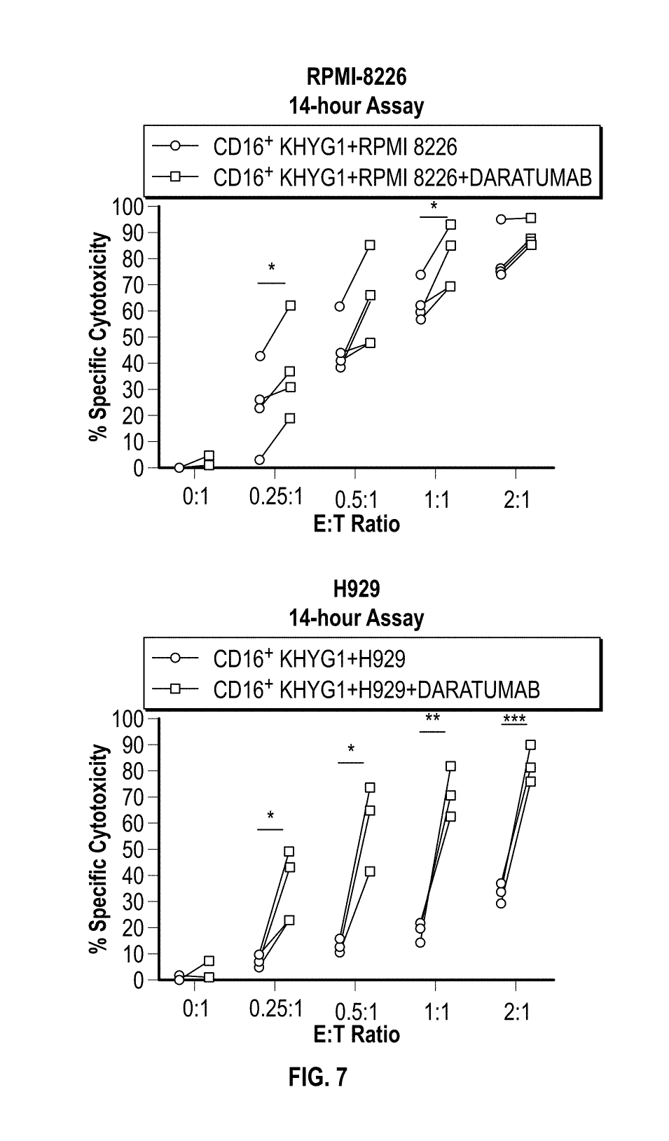

FIGS. 7A-7B show that CD16⁺ KHYG1 NK cells in combination with Daratumumab are more potent in killing CD38^{high} myeloma cell lines at multiple effector:target ratios compared to CD16⁺ KHYG1 NK cells alone.

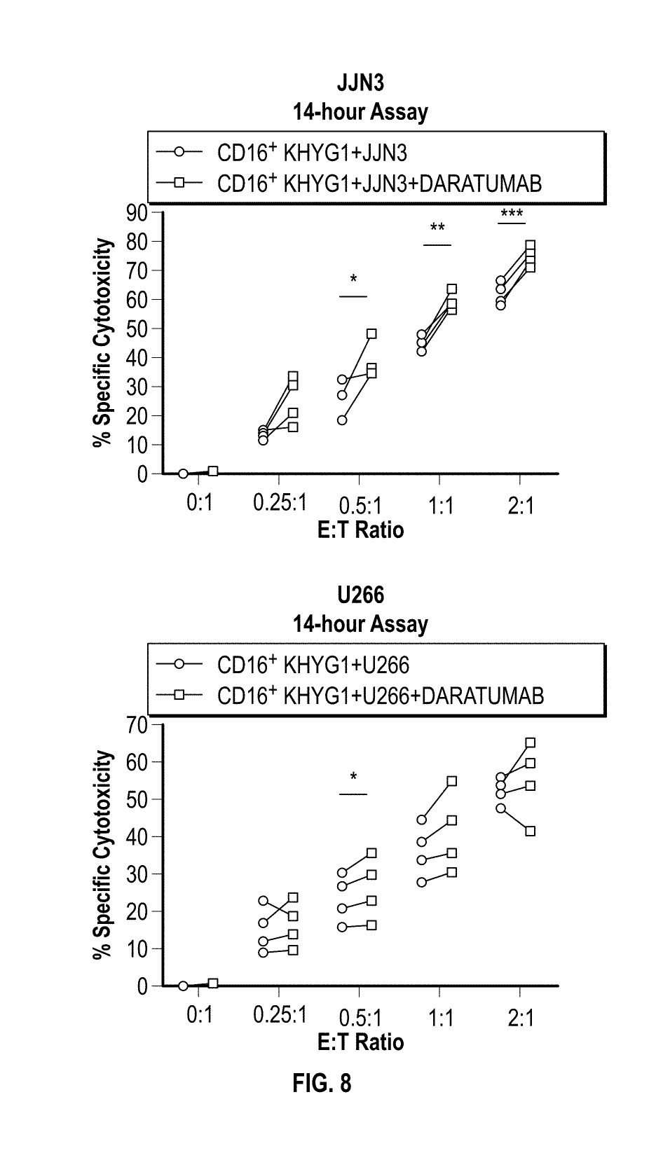

FIGS. 8A-8B show that CD16⁺ KHYG1 NK cells in combination with Daratumumab are more potent in killing CD38^{low} myeloma cell lines at multiple effector:target ratios as compared to CD16⁺ KHYG1 NK cells alone.

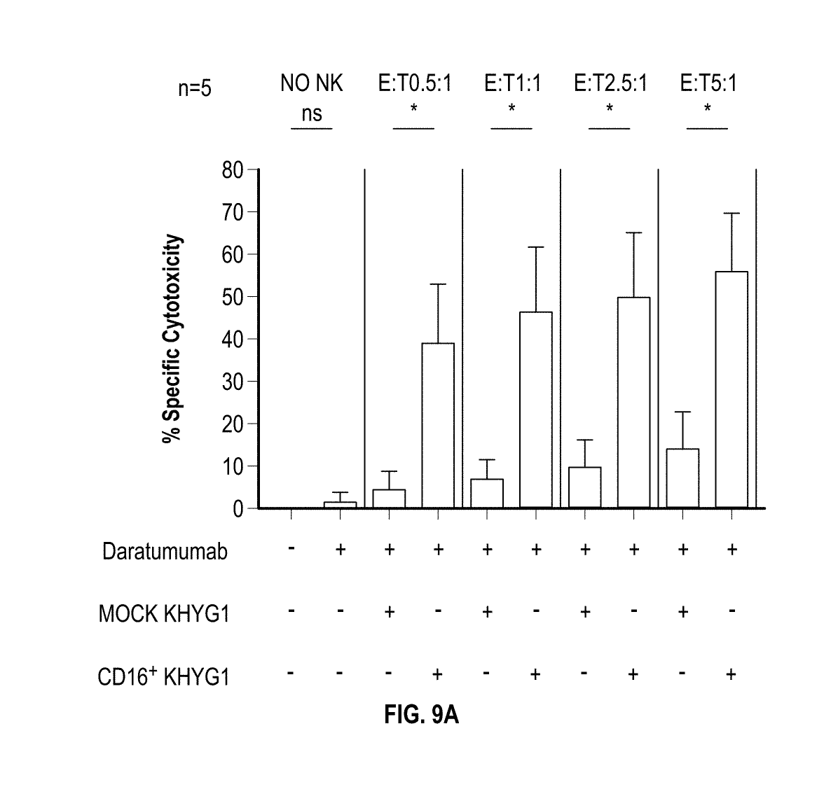

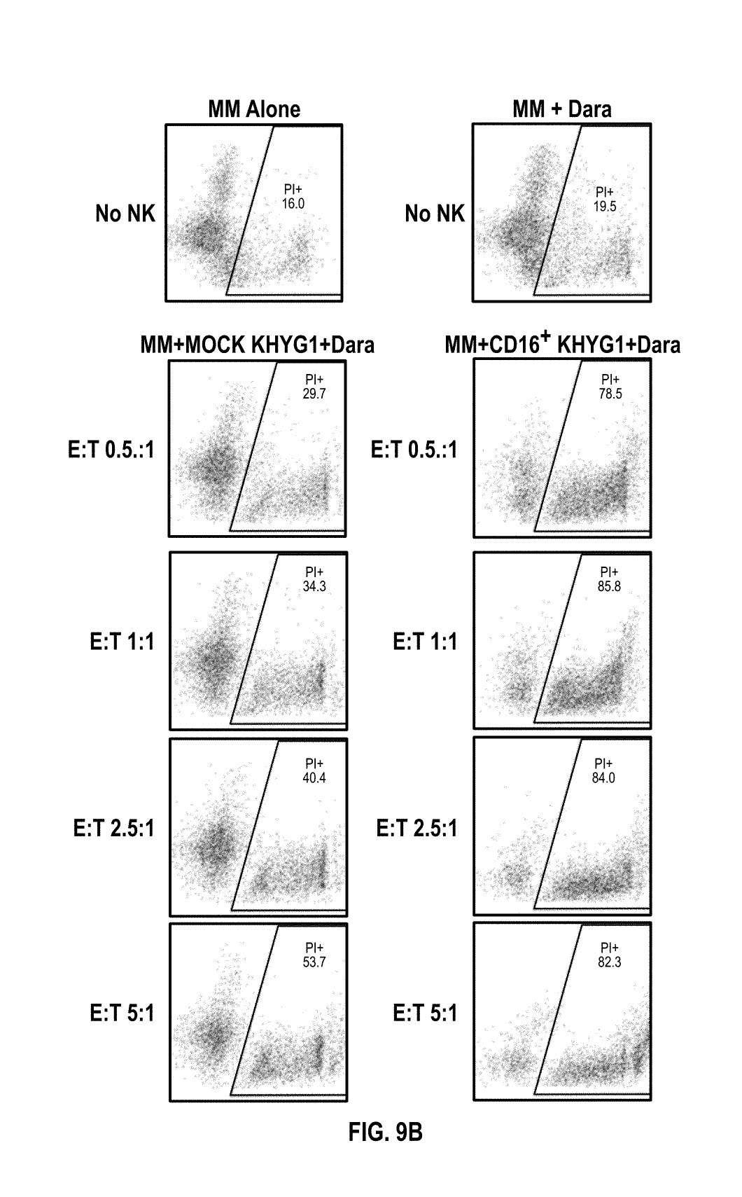

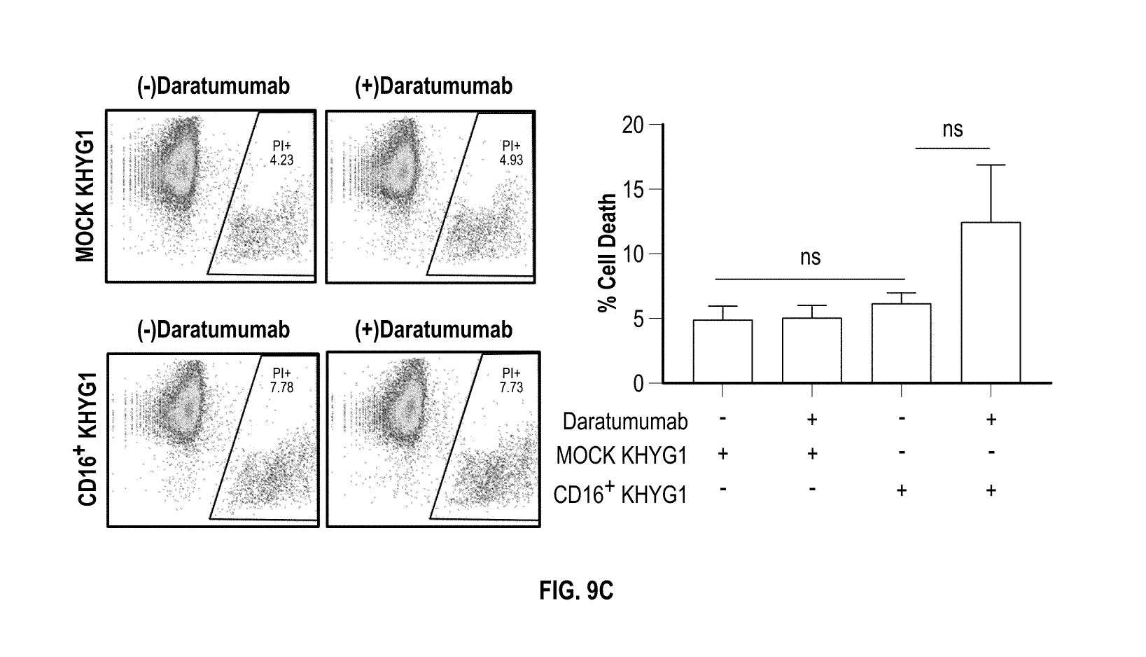

FIGS. 9A-9E show that CD16⁺ KHYG1 NK in combination with Daratumumab eliminate patient derived primary myeloma cells (n=5) more effectively than mock electroporated CD16^{negative} KHYG1 in combination with Daratumumab. FIGS. 9A-9E show the results of a 14-hour ADCC assay with mock nucleofected or CD16 mRNA nucleofected KHYG1 against primary multiple myeloma (MM) cells in combination with Daratumumab. To obtain the results shown in this figure primary MM cells from 5 patients were independently tested and the data then pooled.

FIGS. 9F-9G show the discernible, but non-significant increase in Daratumumab induced NK cell fratricide on CD38^{low} CD16 expressing KHYG1 in the absence of any target cells. Greater than 80% of the genetically modified cells are viable for experimental purposes.

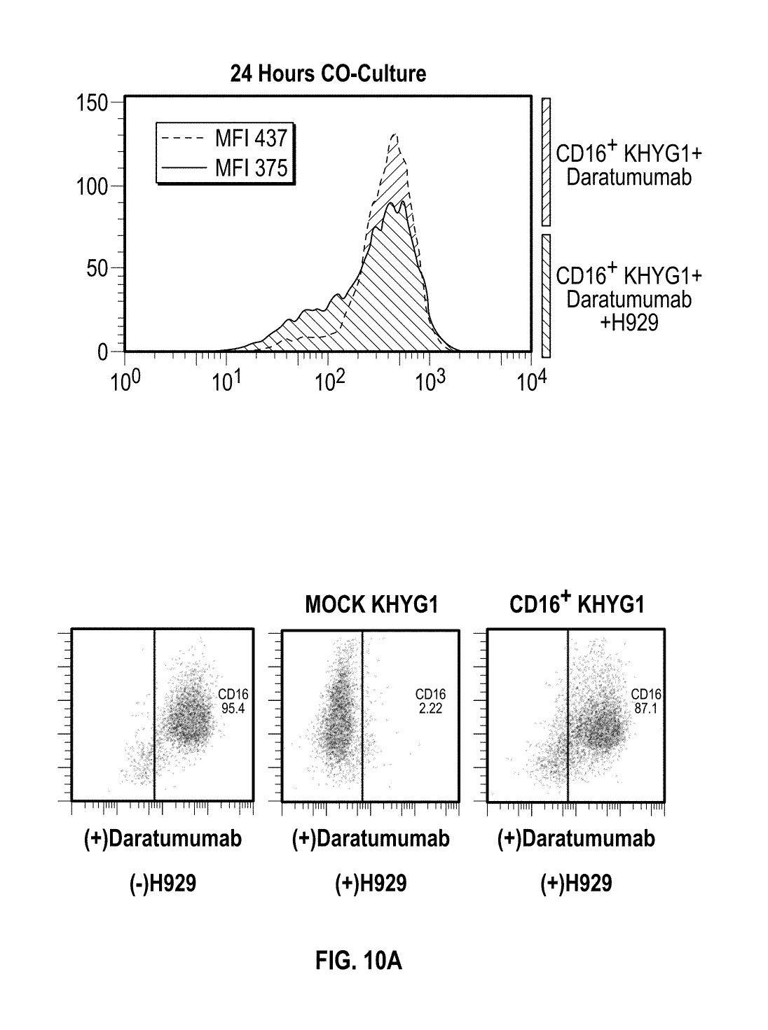

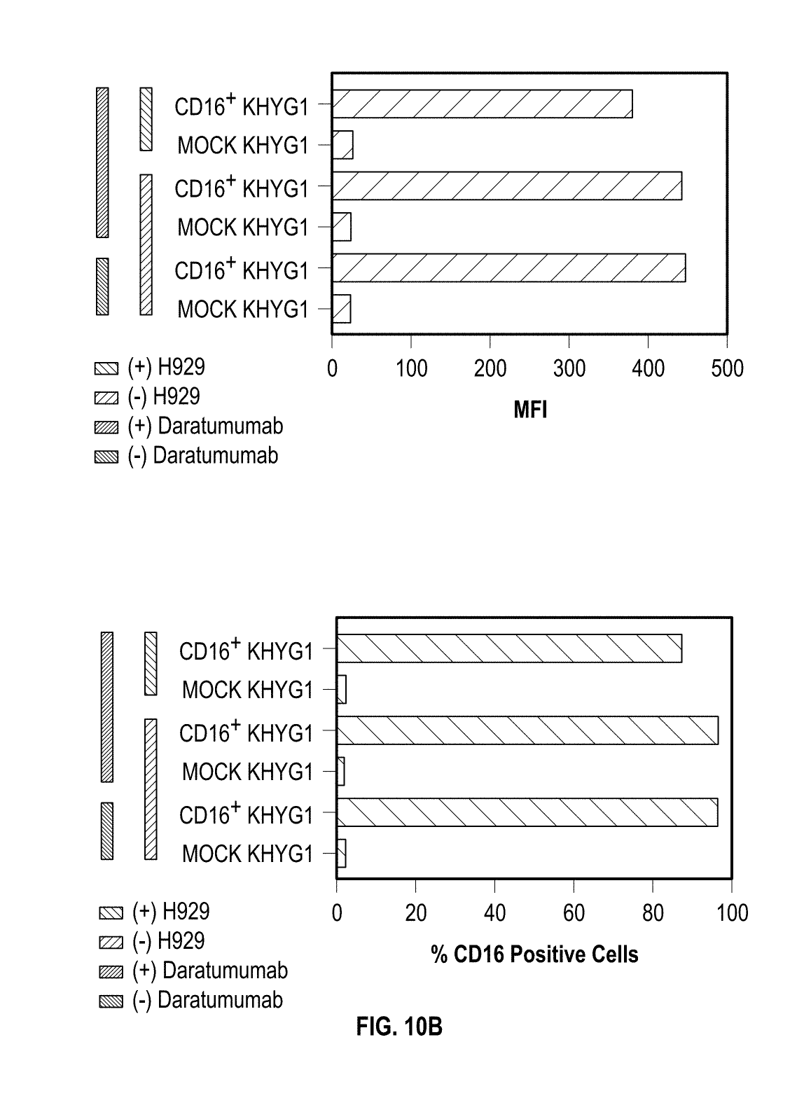

FIGS. 10A-10D show that CD16⁺ KHYG1 NK in combination with Daratumumab exhibit less shedding of the CD16 receptor upon interaction with cells of the H929 (multiple myeloma) cell line.

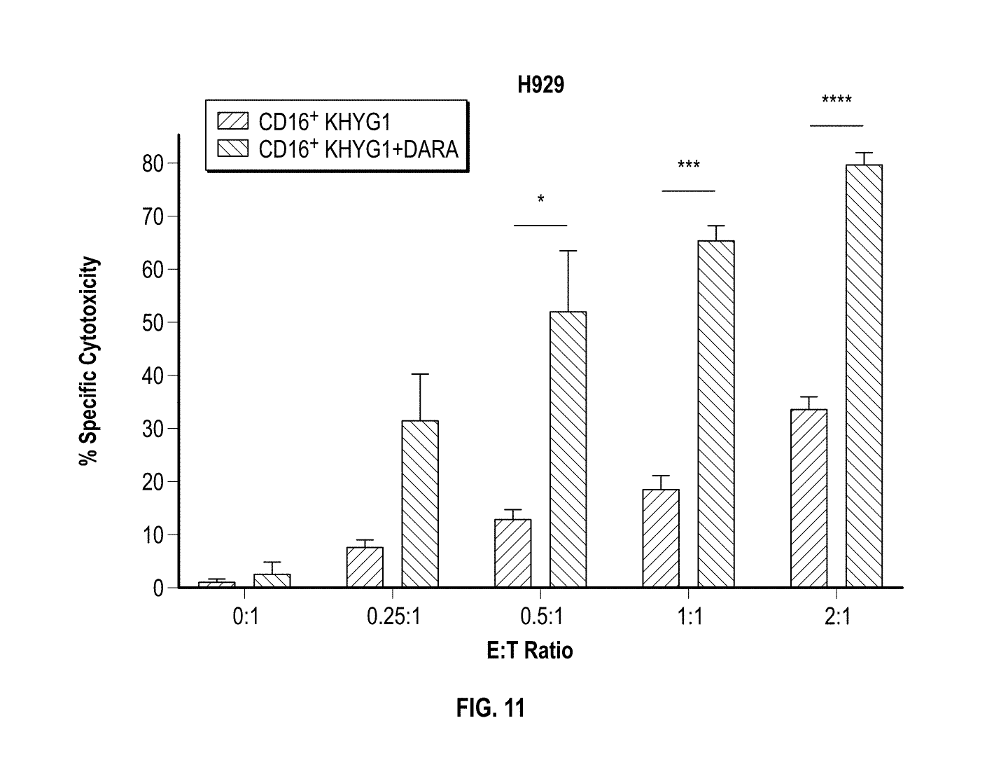

FIGS. 11 shows the results of a 14-hour ADCC assay with CD16 mRNA nucleofected KHYG1 against H929 cells with or without Daratumumab.

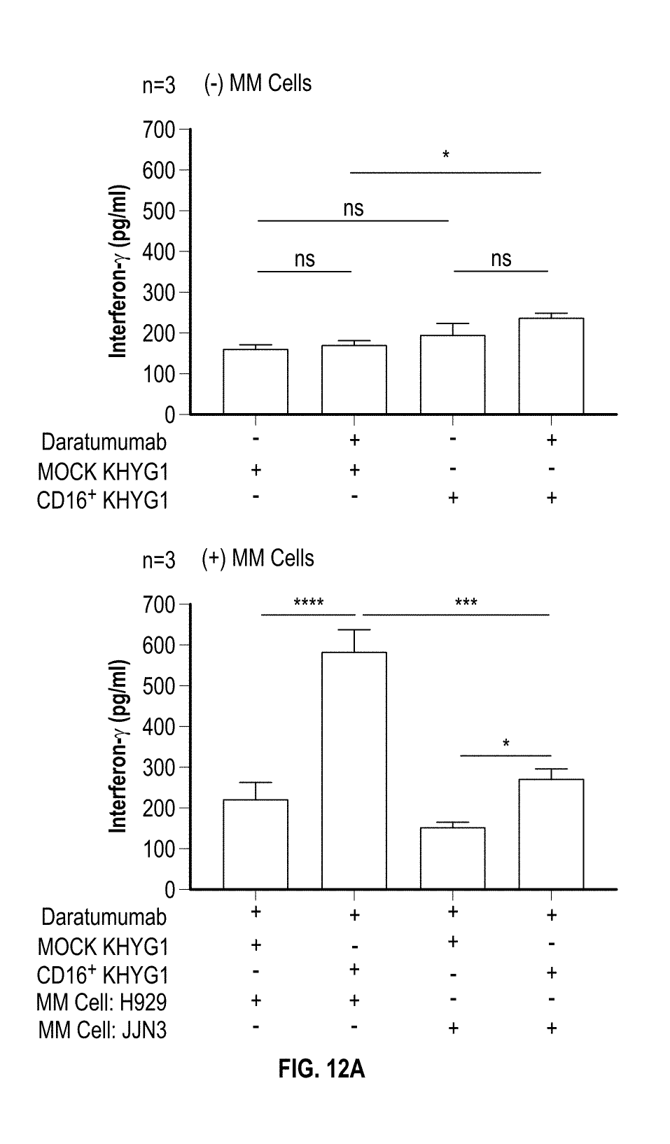

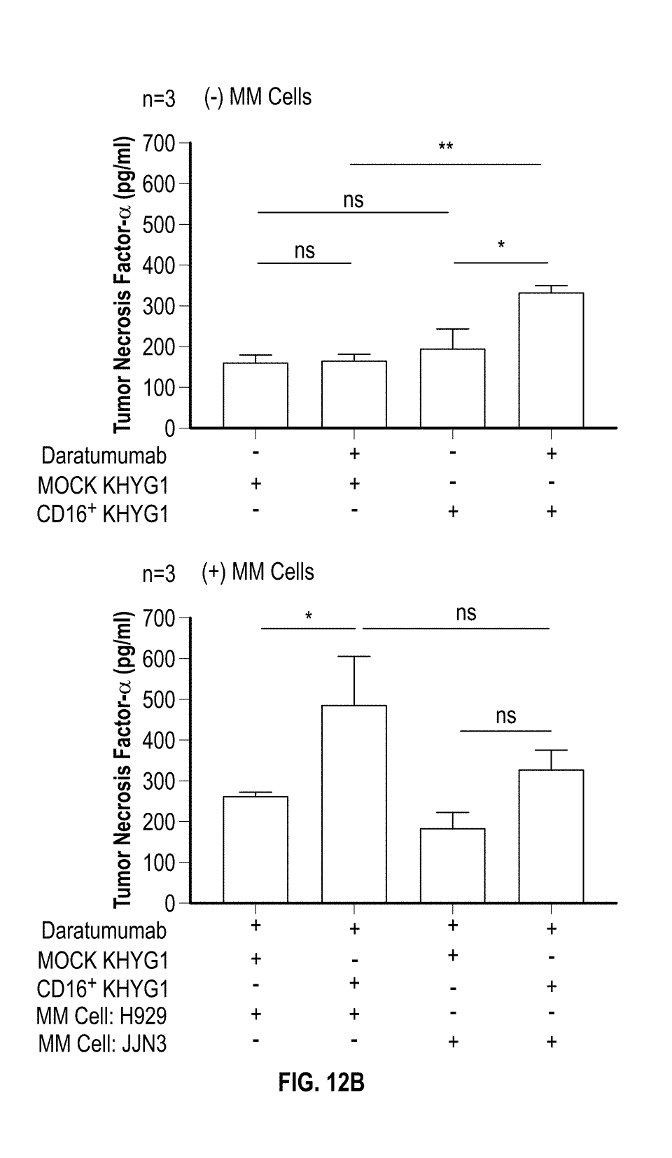

FIGS. 12A-12D show cytokine release during a 14-hour NK cell-MM cell co-culture measured by ELISA for (a) interferon gamma (IFNγ), and (b) TNF-α. Mock nucleofected or CD16 m-RNA nucleofected KHYG1 cells were co-cultured with MM cell lines, in the presence of Daratumumab. As control, mock nucleofected or CD16 m-RNA nucleofected KHYG1 cells were cultured alone in absence of any target tumor cells in presence or absence or Daratumumab to determine if the genetic modification alone induced cytokine production in the absence of any relevant target tumor cells. Standard curves for the ELISA assays were carried out and it was confirmed that the quantities measured for the experiments presented fell within the linear range of the ELISA.

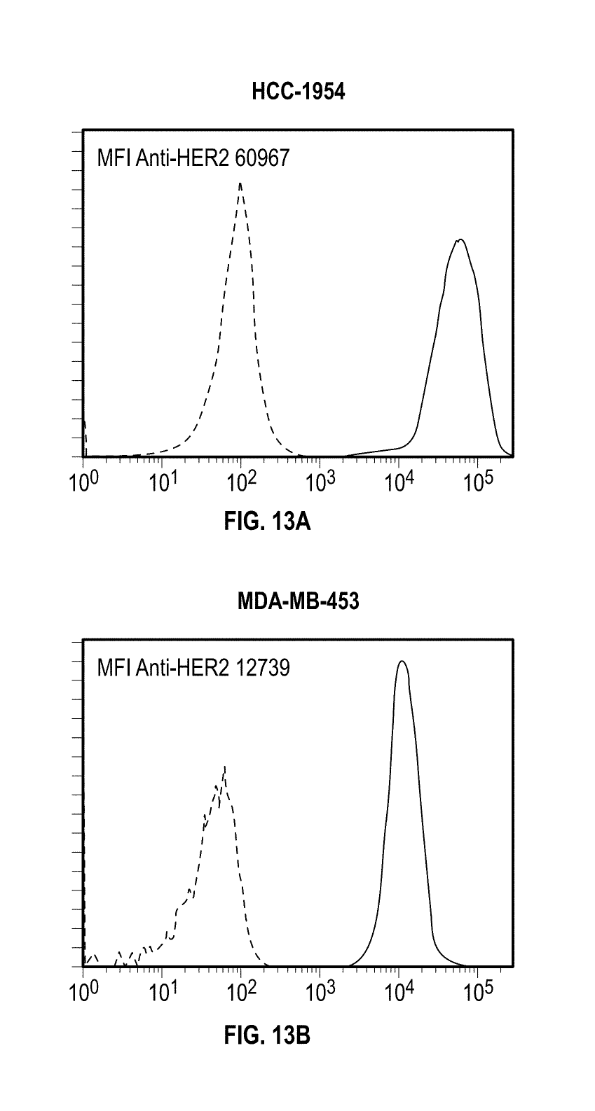

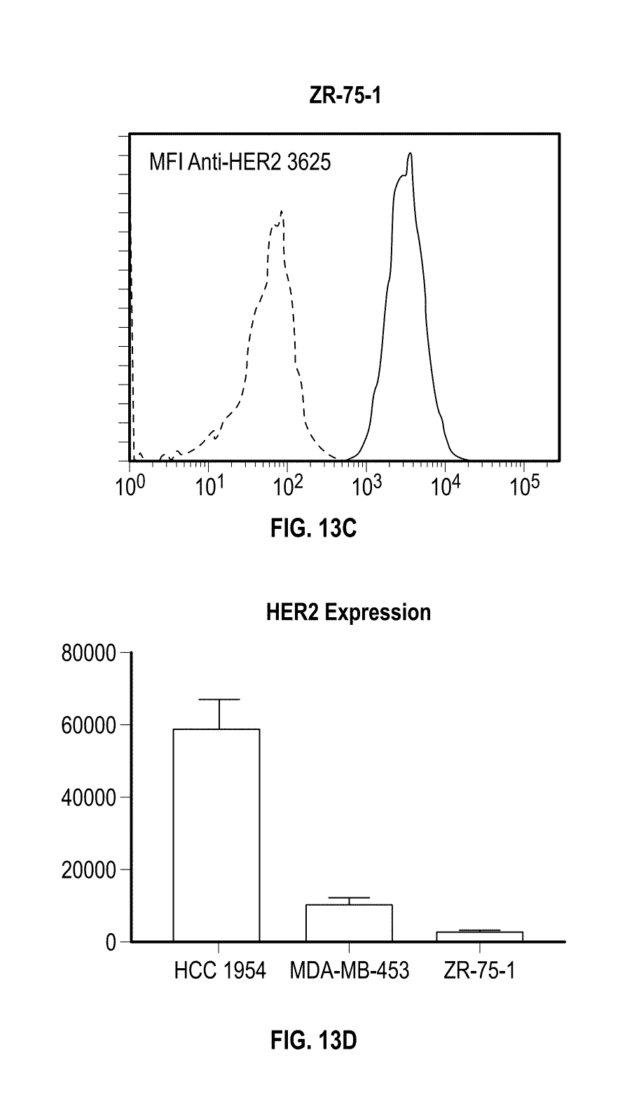

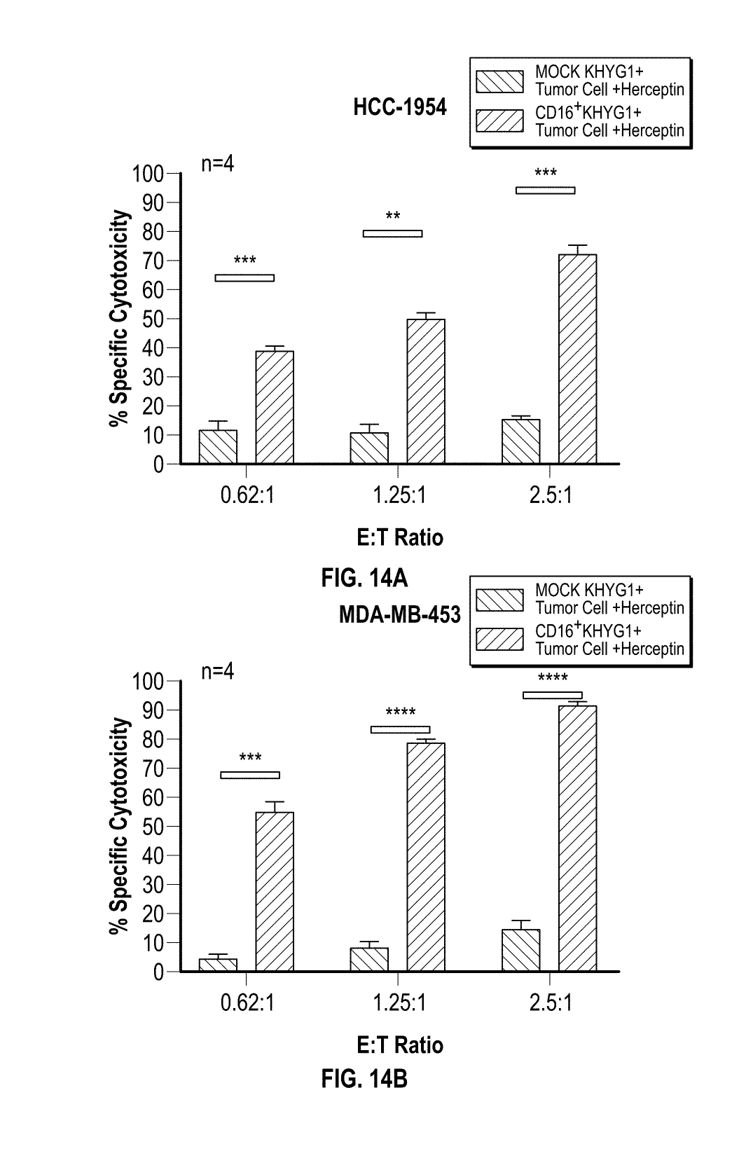

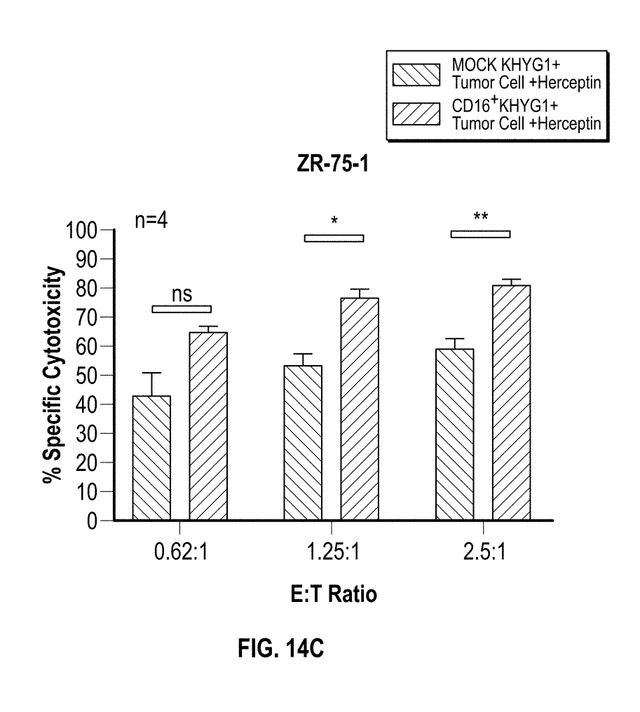

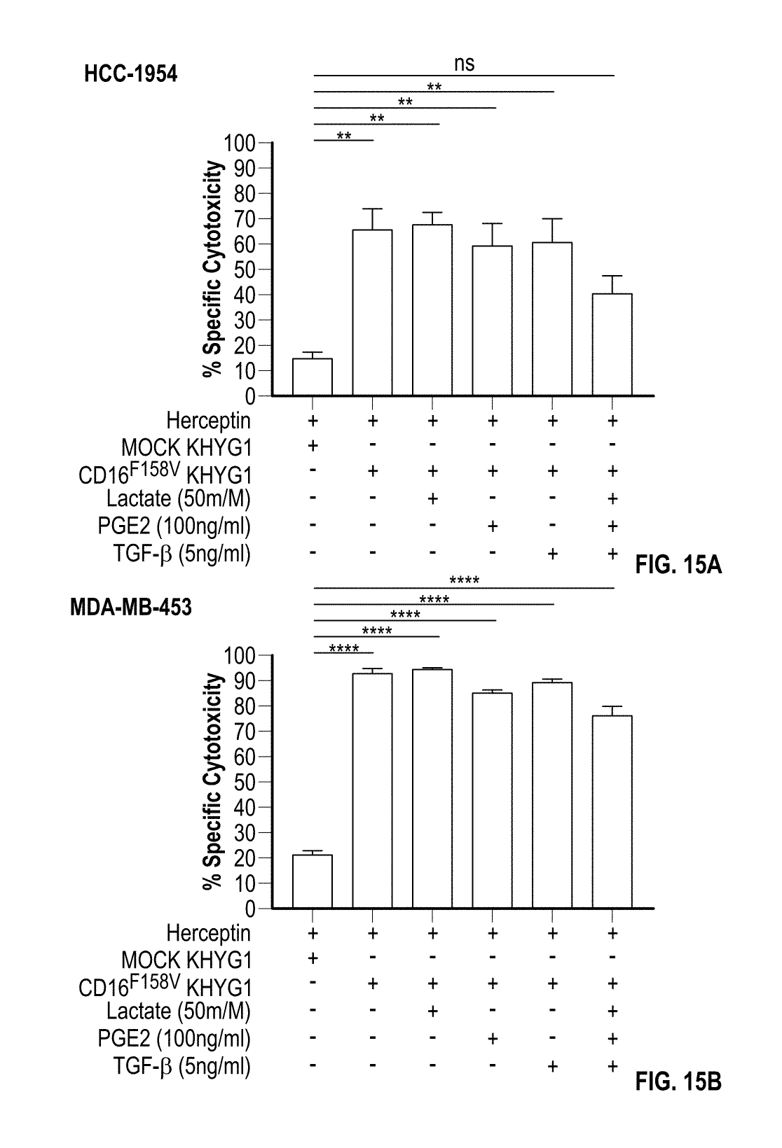

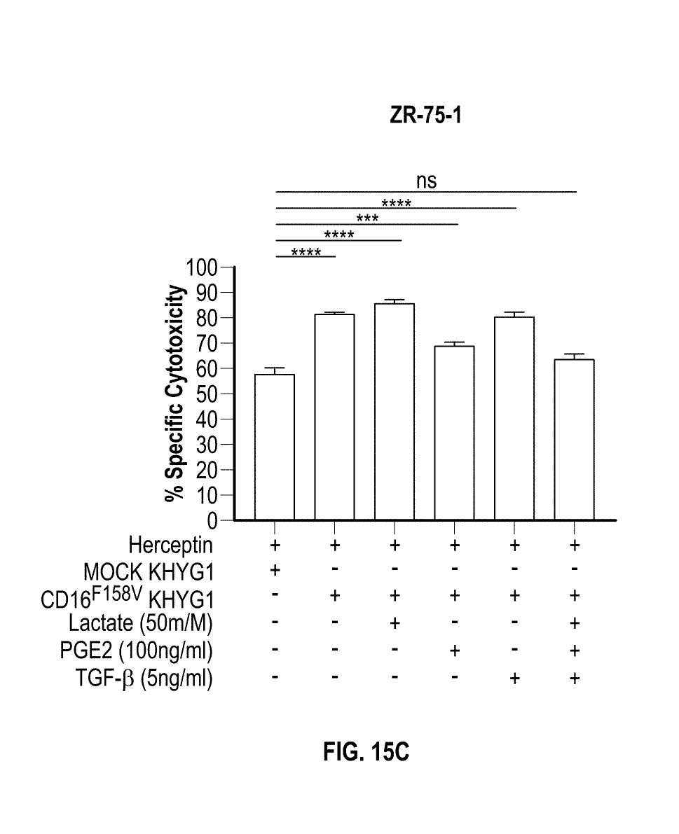

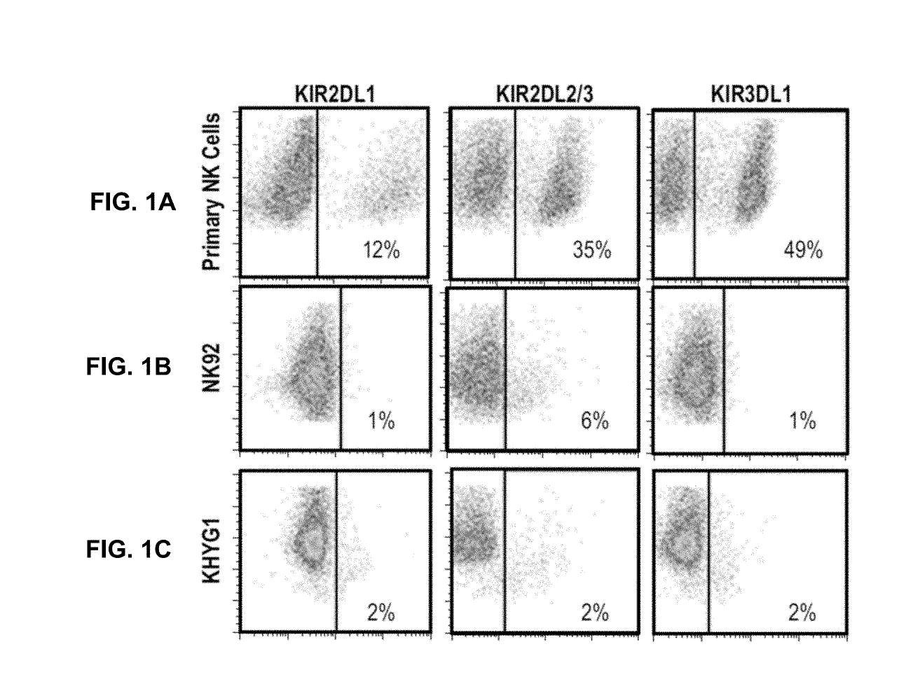

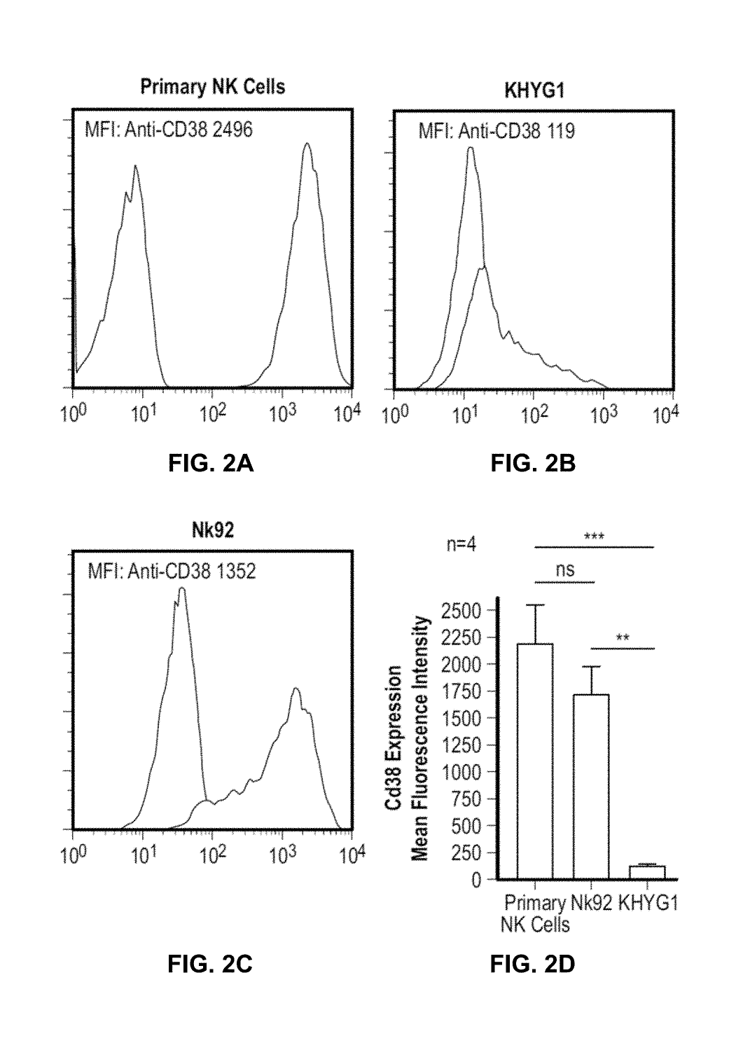

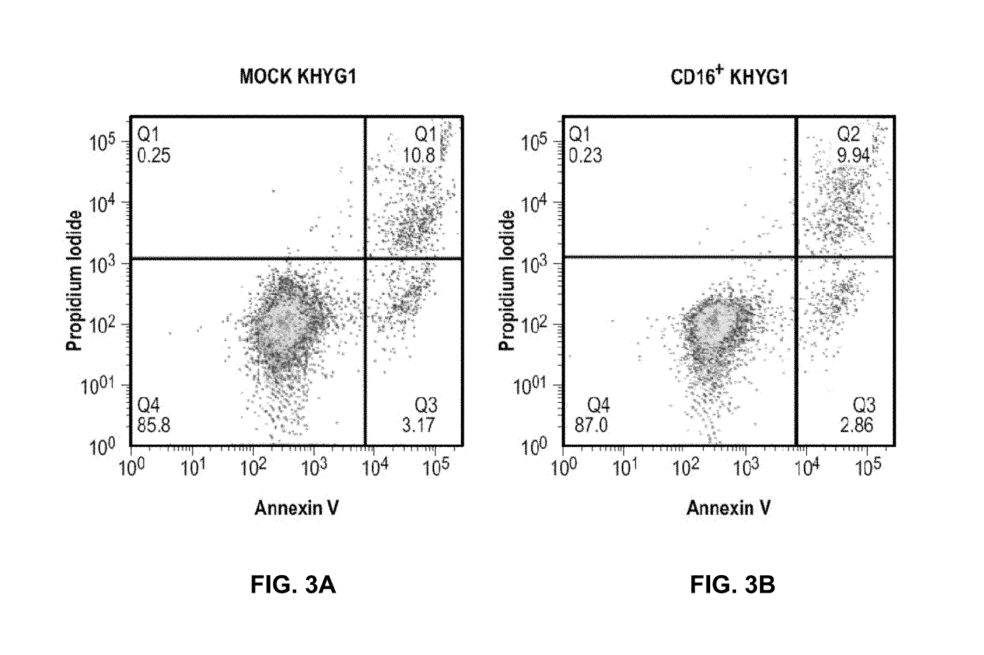

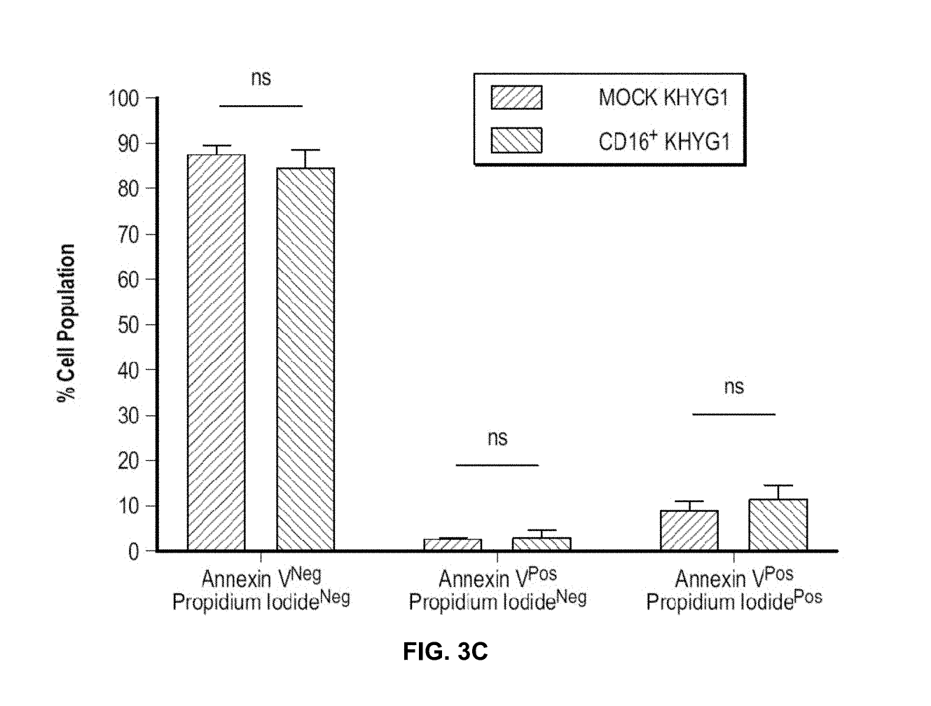

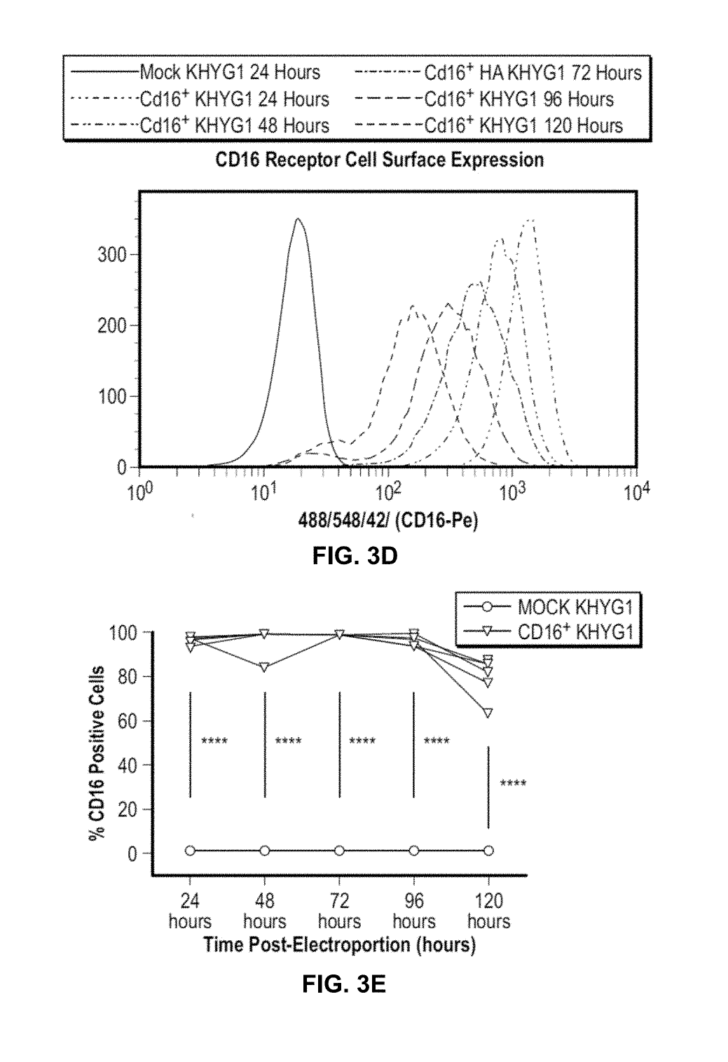

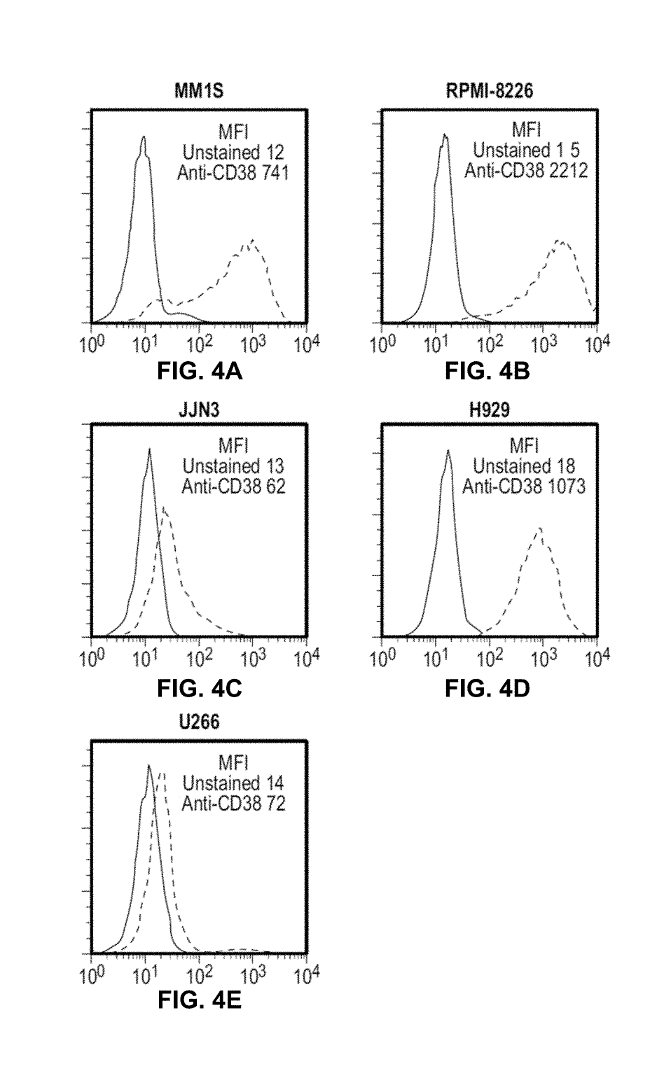

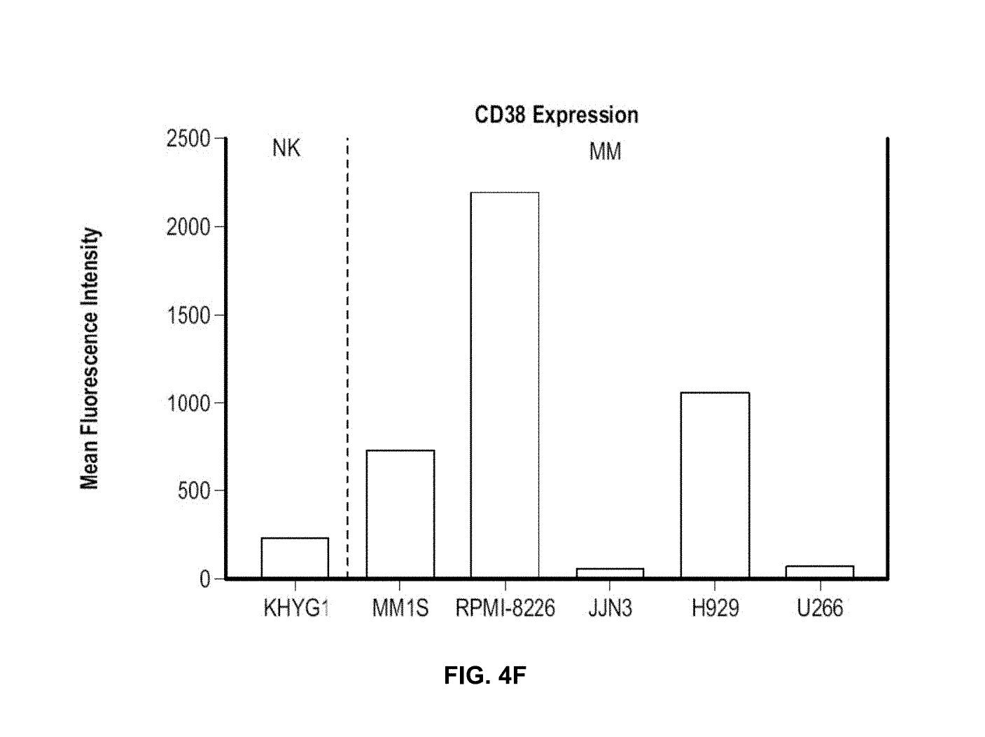

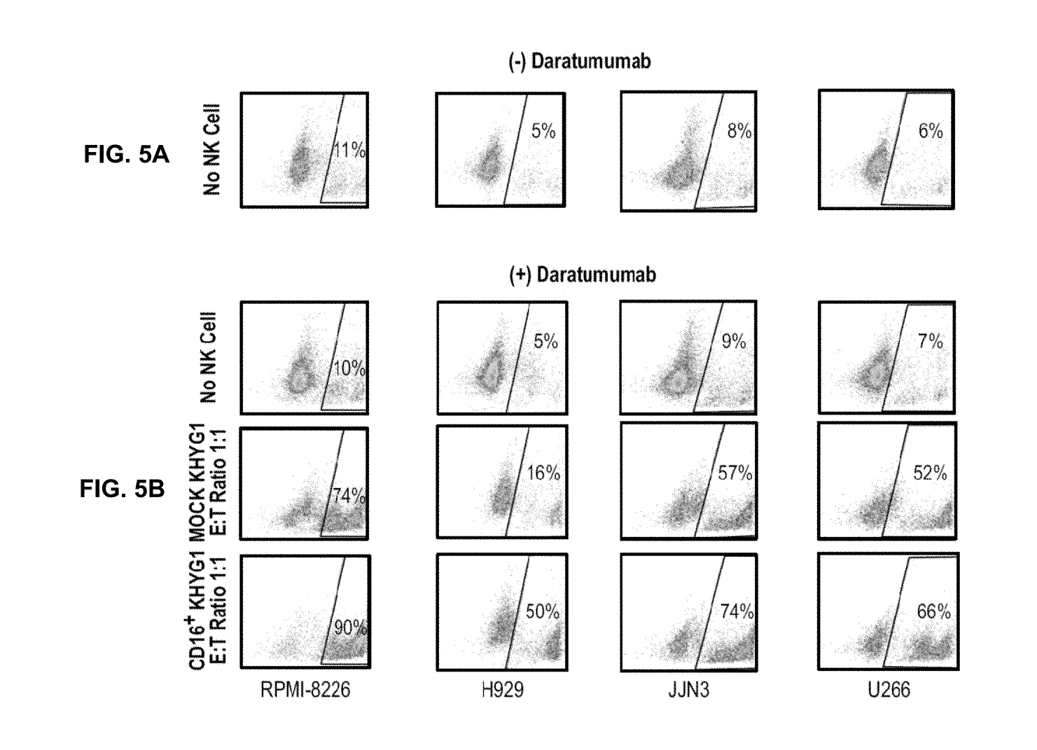

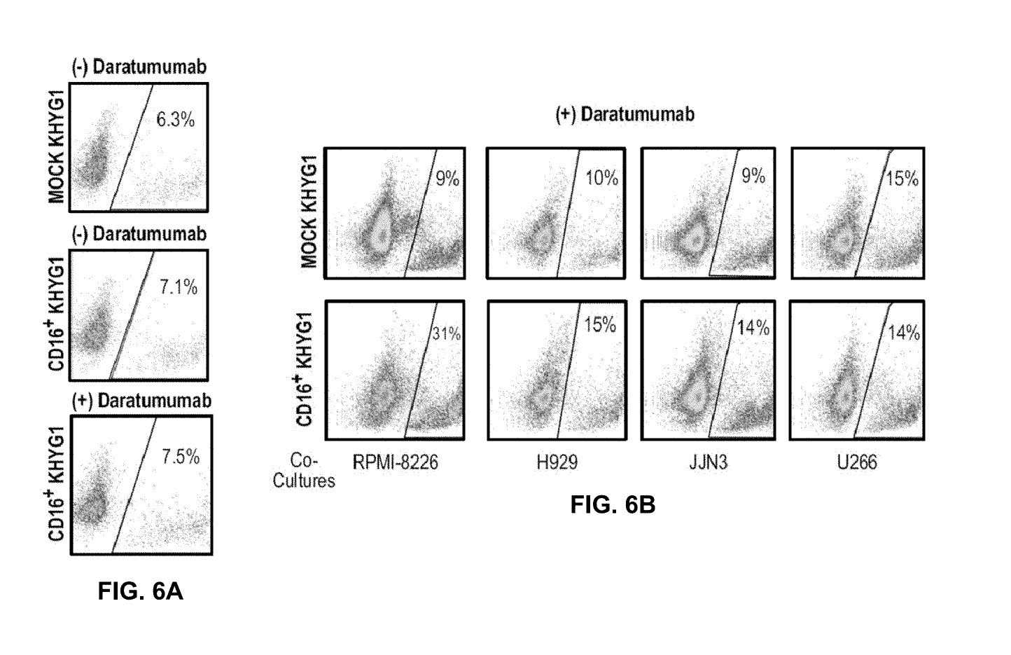

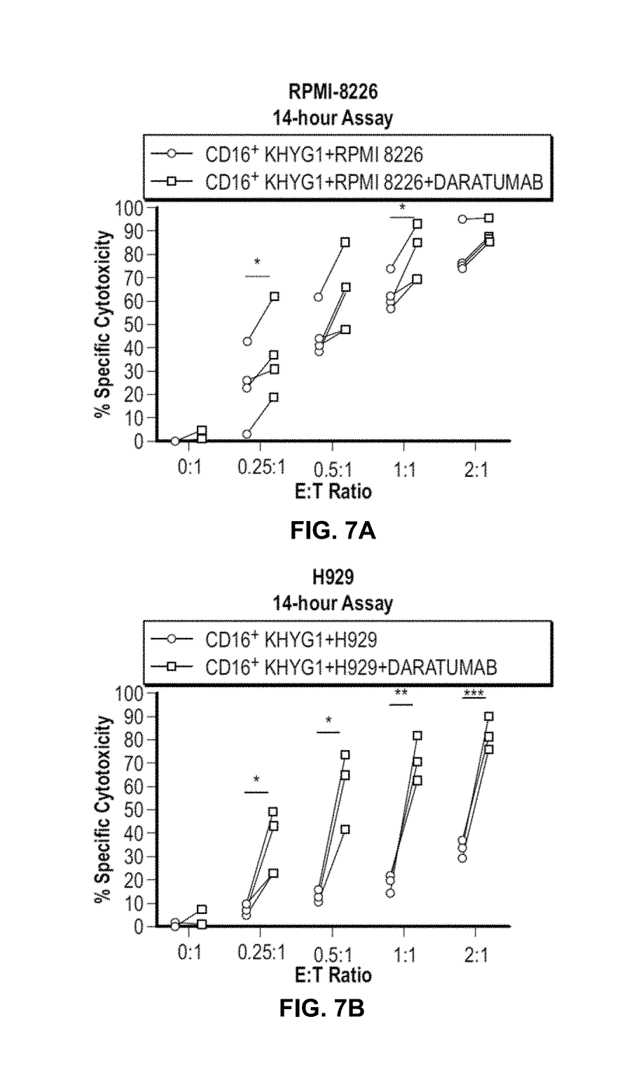

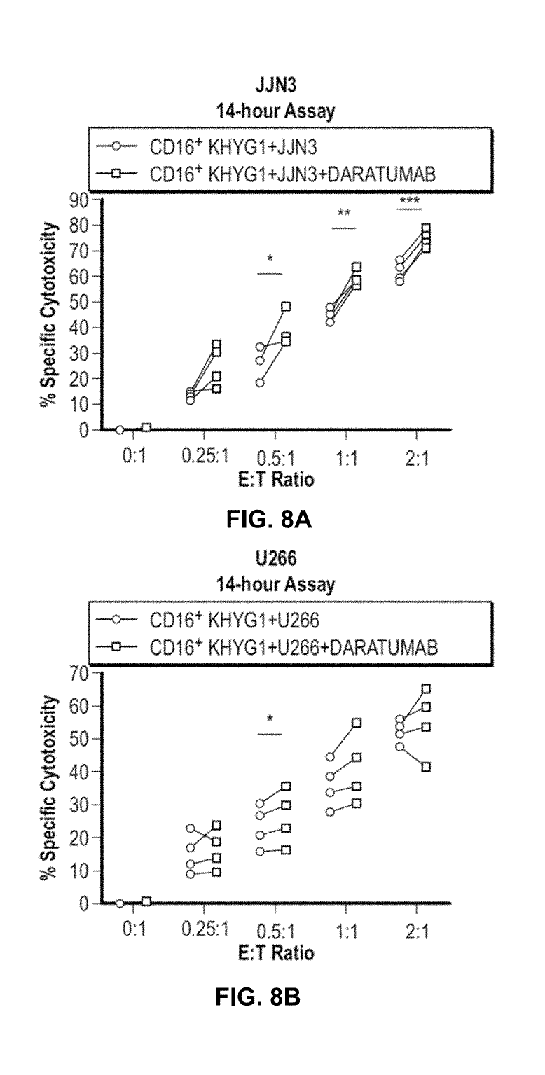

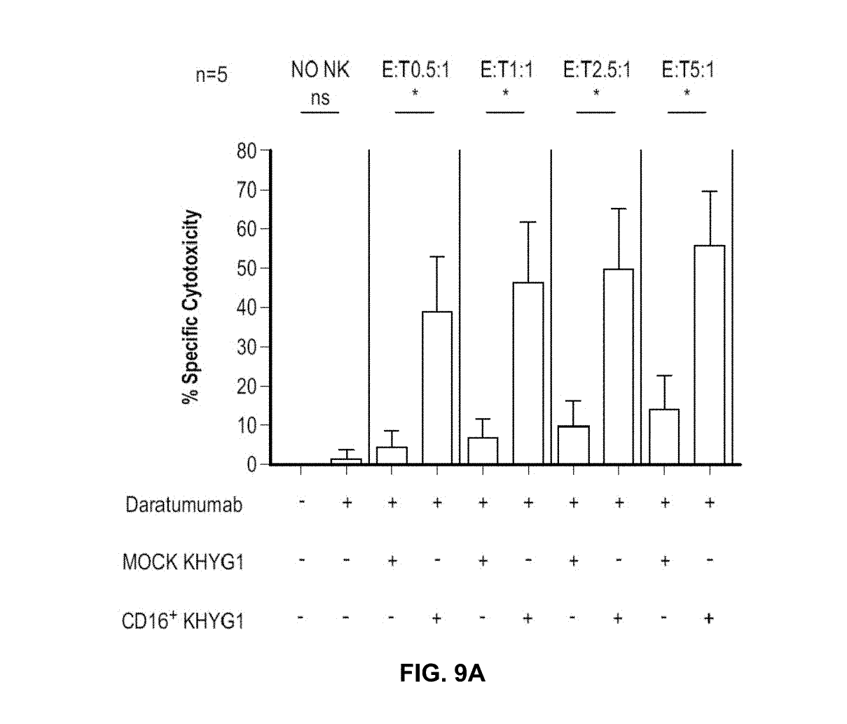

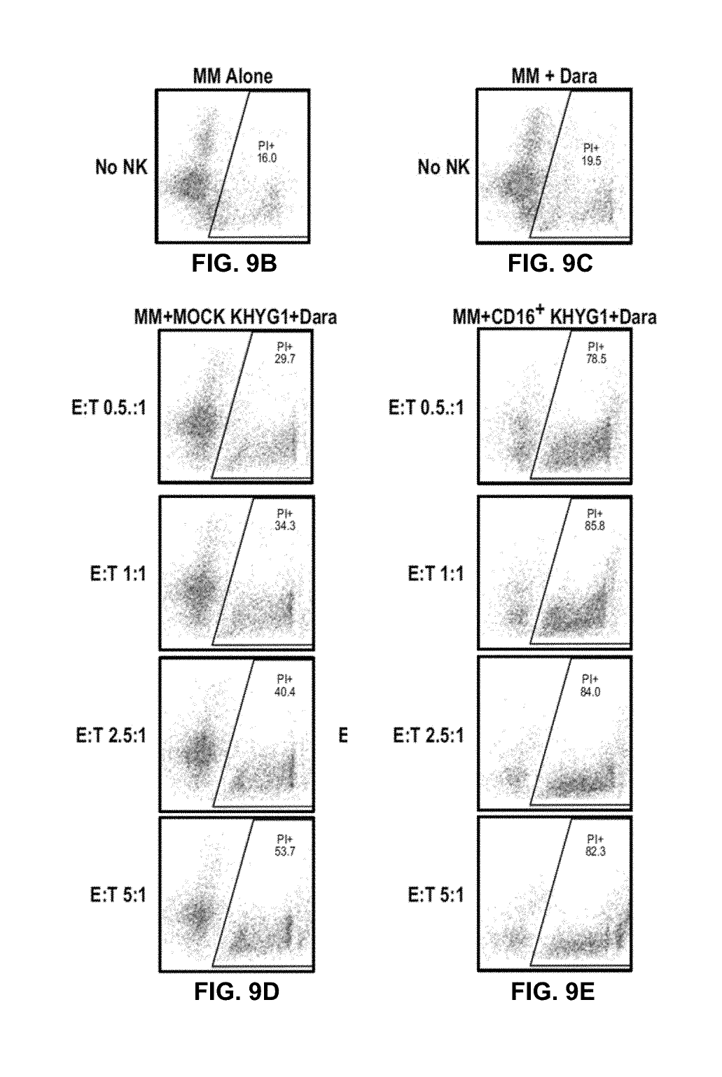

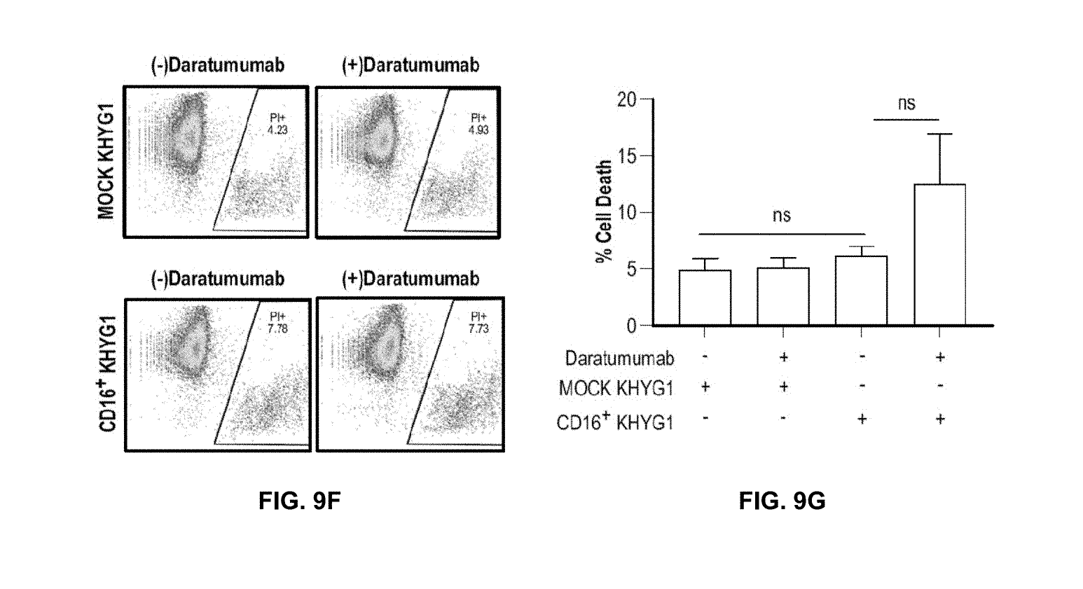

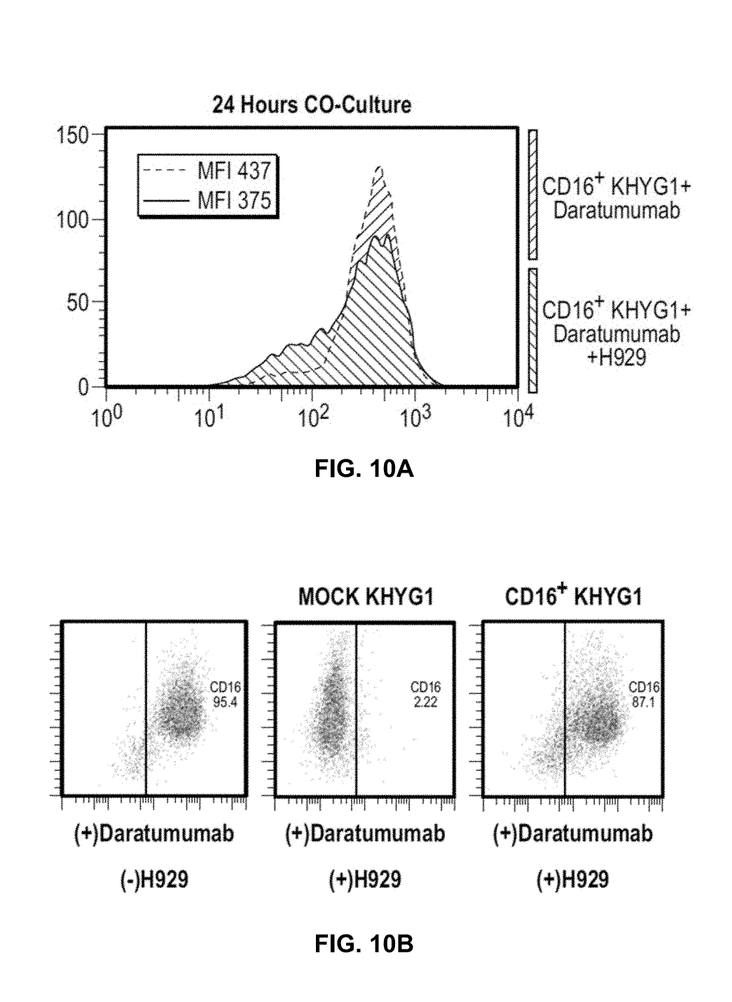

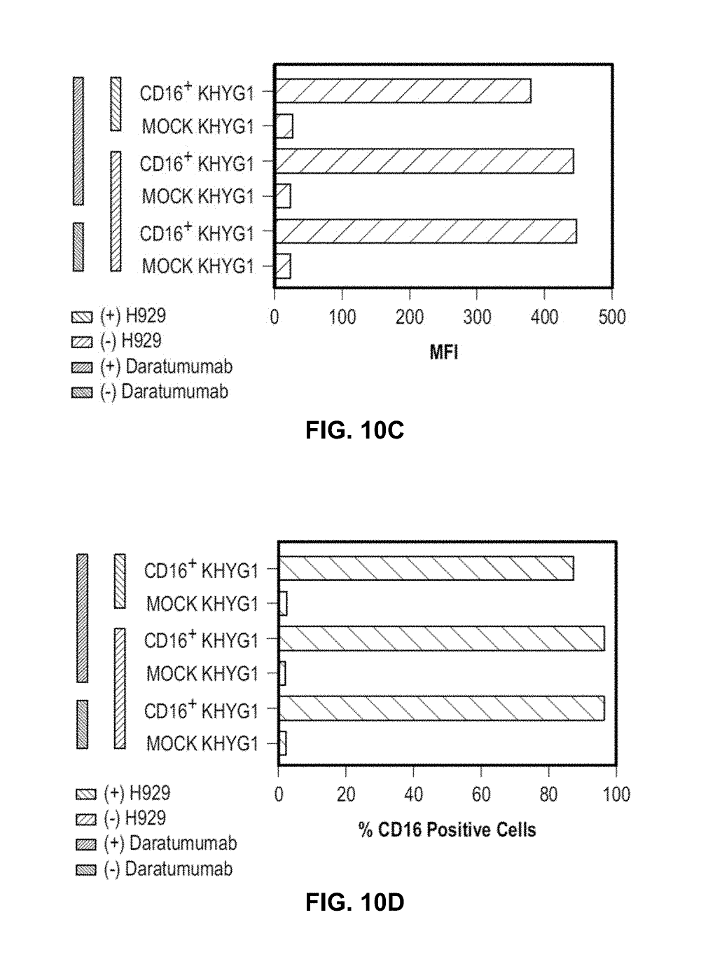

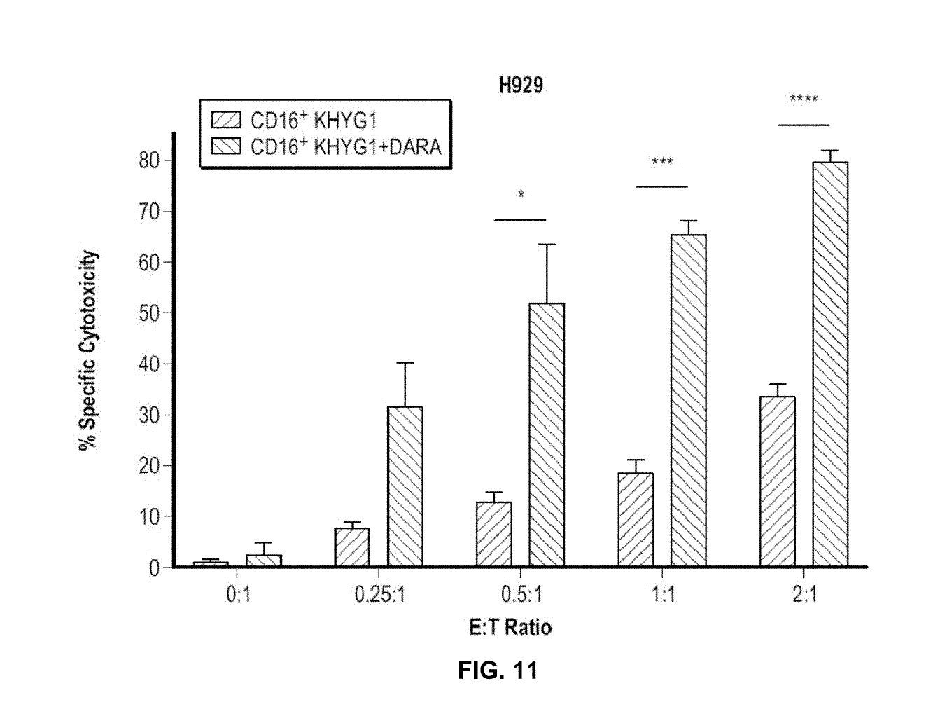

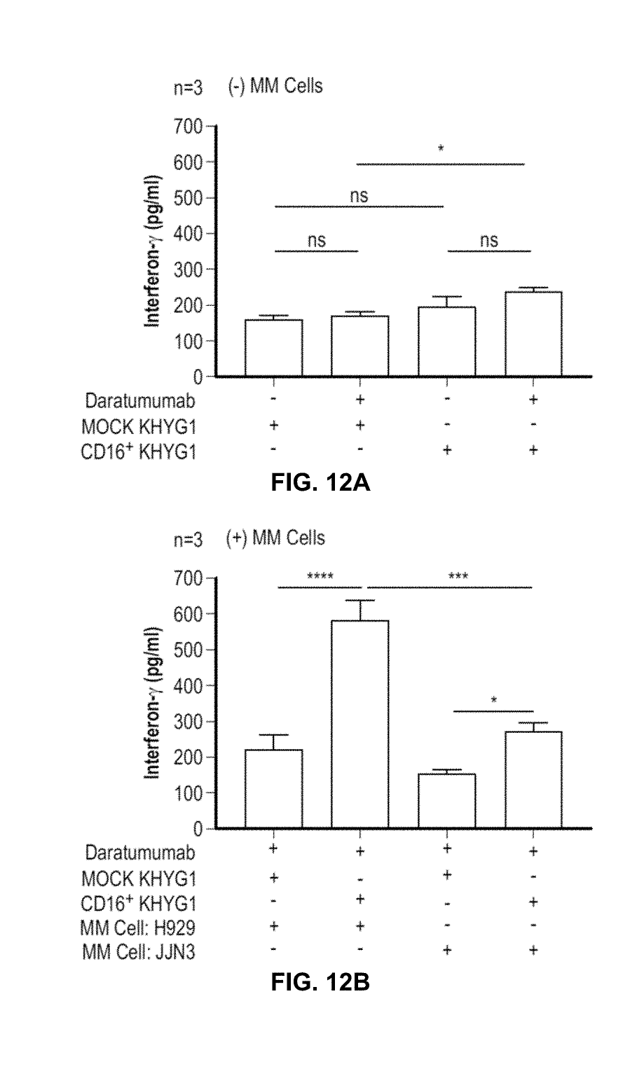

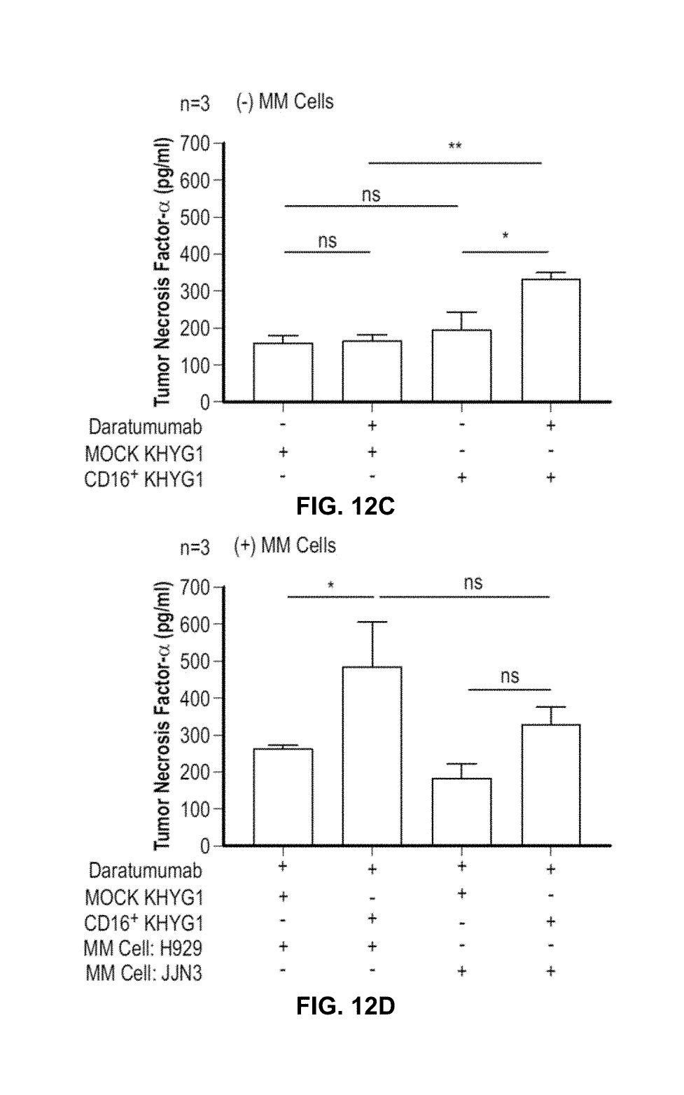

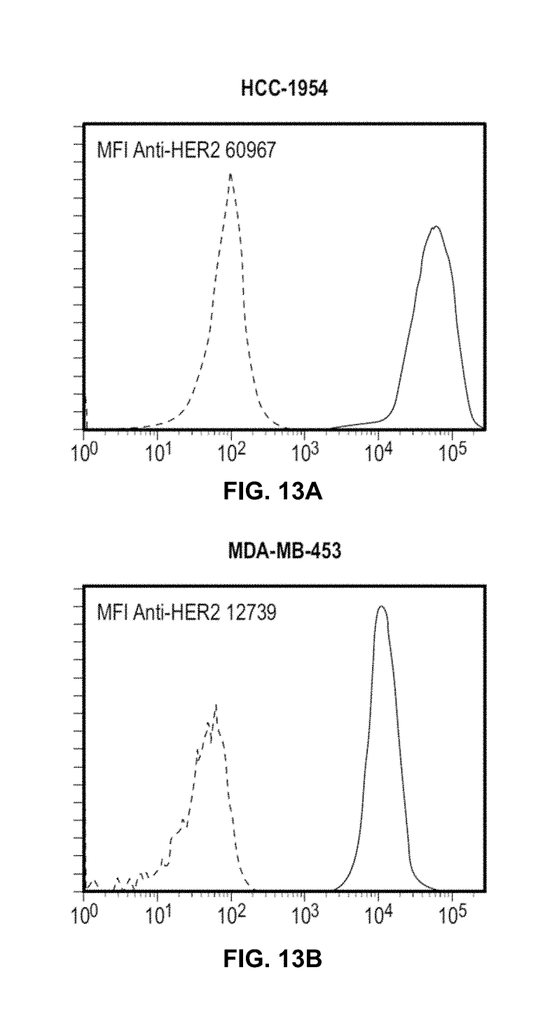

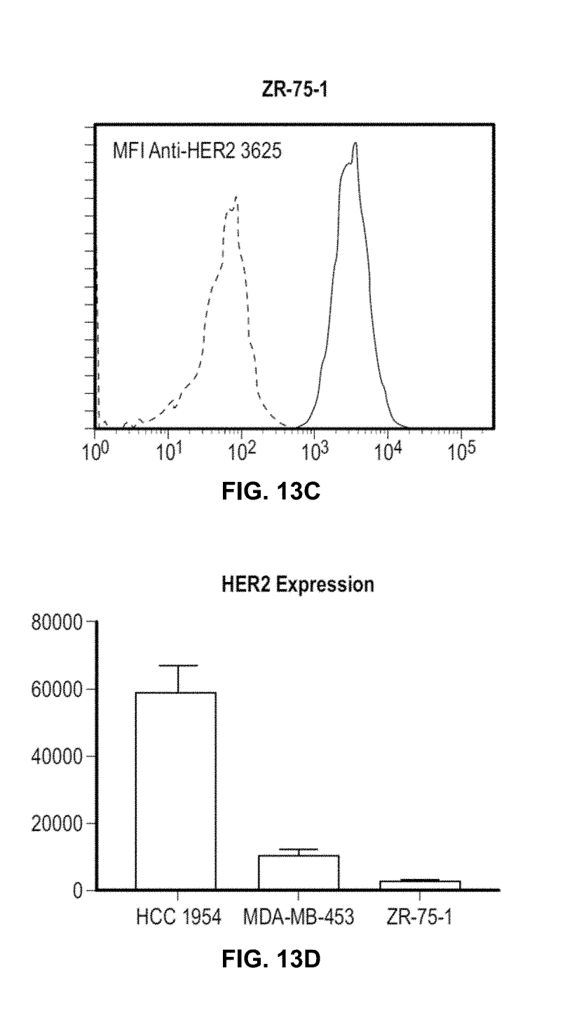

FIGS. 13A-13D show expression levels of HER2 in 3 breast cancer cell lines.

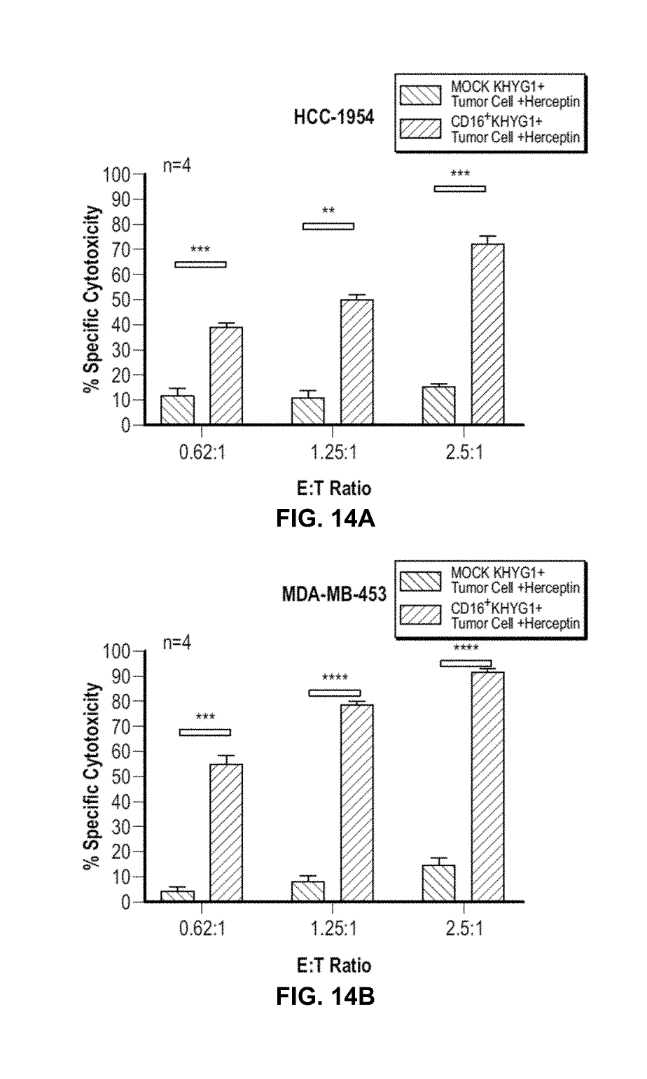

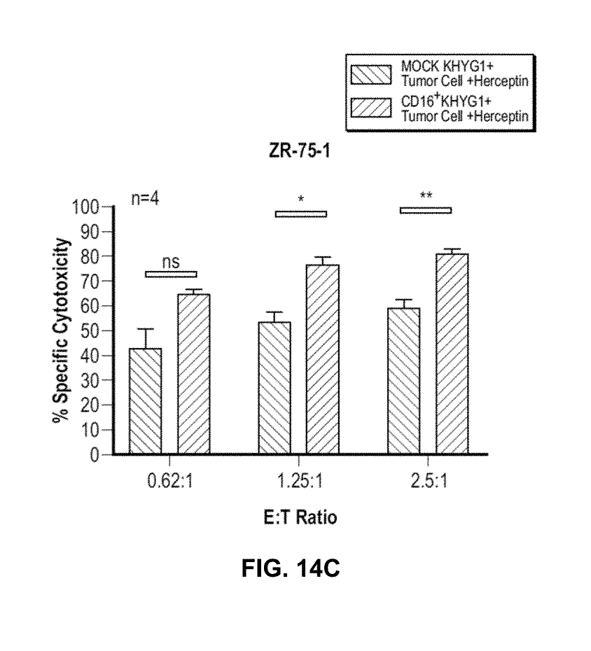

FIGS. 14A-14C show the cytotoxicity of mock KHYG1 cells vs CD16 mRNA electroporated KHYG1 cells against 3 breast cancer cell lines in an ADCC assay.

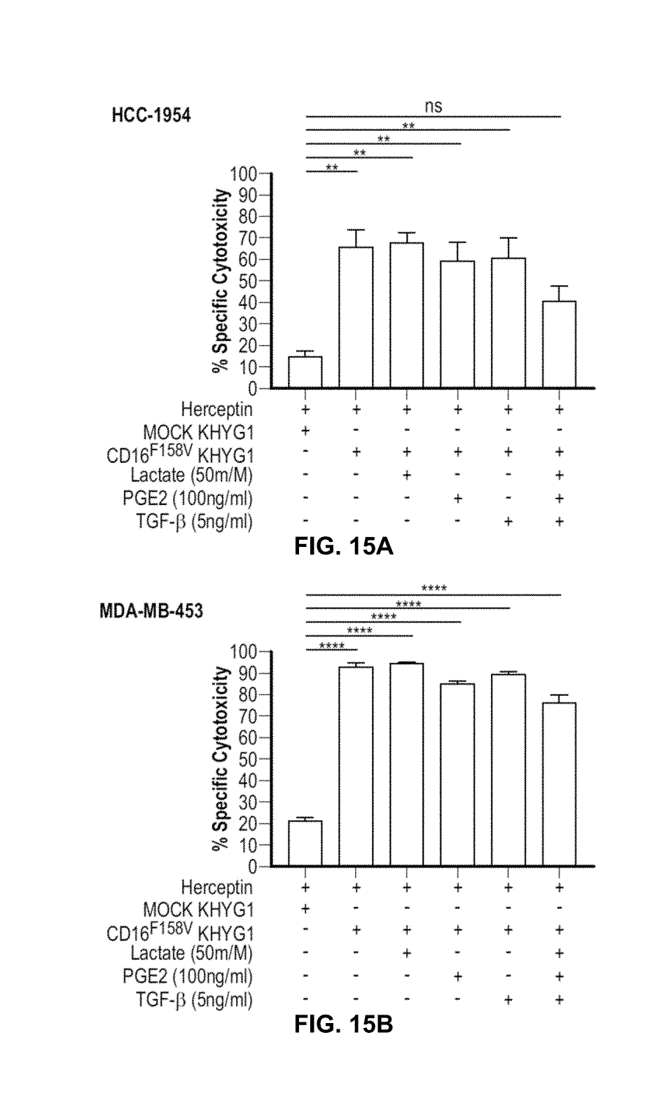

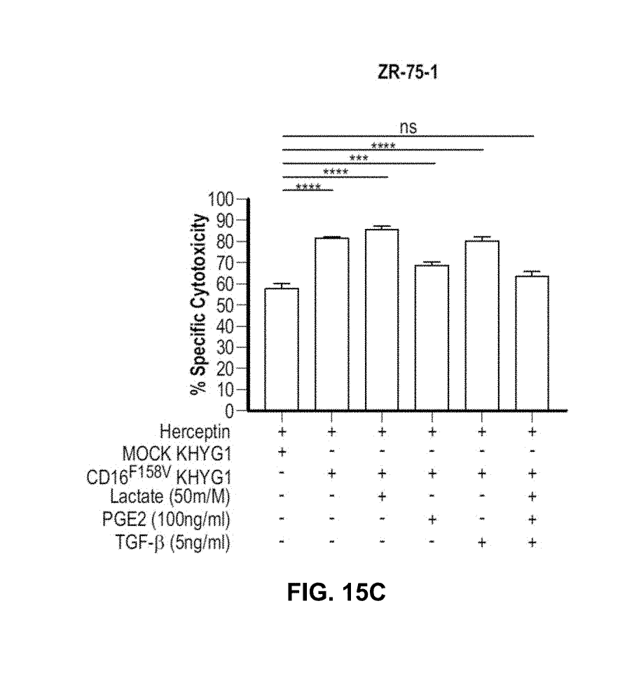

FIGS. 15A-15C show the ability of CD16 mRNA electroporated KHYG1 cells to maintain cytotoxicity against 3 breast cancer cell lines in the presence of immunosuppressive factors.

### Example 1

### Measuring the Level of CD38 Expression on Multiple Myeloma and NK Cell Lines

CD38 expression was determined for a panel of cell lines: RPMI-8226, H929, MM.1S, U266 and JJN3 by employing the staining protocol for CD38 expression as set out below. The stained cells were then analysed by flow cytometry (FACS). The results of these experiments are shown in FIGS. 4A-4F (which are representative of 4 separate experiments) and reveal that multiple myeloma cell lines have a broad-spectrum cell surface expression of CD38. The expression of CD38 on KHYG1 cells was low in comparison with at least cell lines MM.1S, RPMI 8226 and H929.

### Example 2

### KYHG1 as a Candidate NK Cell for CD16 Expression

CD38 and KIR expression was determined for expanded primary NK cells, and cell lines NK92 and KHYG1 by employing the staining protocols for CD38 and KIR expression as set out below. The stained cells were then analysed by flow cytometry (FACS). The results of these experiments are shown in FIGS. 1A-1C and FIGS. 2A-2D. The expression of both CD38 and the KIR inhibitory receptors is much lower in KHYG1 in comparison to the mean fluorescence intensities and expression levels seen for NK92 and expanded primary NK cells.

### Example 3

### Assessing the Viability and Kinetics of CD16 m-RNA Electroporated KHYG1 NK Cells

mRNA transcripts coding for high affinity (HA) CD16 protein were synthesized using in vitro transcription (IVT), and KHYG1 cells were subsequently electroporated with the CD16 mRNA according to the protocol for Electroporation of CD16 mRNA into KHYG1 NK cells and time course experiments that are set out below. The results of this experiment are shown in FIGS. 3A-3E and show that CD38^{low} KHYG1 NK cells can transiently overexpress CD16 receptor over a period of 120 hours.

### Example 4

### Demonstration that CD16 Expressing KHYG1 Induces Cell Death in Daratumumab Treated Multiple Myeloma Cells

CD16 expressing KHYG1 cells were analyzed for surface expression of CD16, and further co-cultured with multiple myeloma cell lines RMPI 8226, H929, JJN3 and U266 either alone or in combination with Daramutumab in an ADCC assay. NK cell induced cytotoxicity was measured by FACS-based methods as described below. The results are shown in FIGS. 5A-5B. The boxed panel to the right of the FACS frequency plots indicates gating for dead cells as determined by propidium iodide staining.

HA CD16 nucleofected KHYG1 in combination with Daramutumab was significantly more cytotoxic towards NK resistant multiple myeloma cell lines JJN3 and H929; data represents mean of 4 independent experiments) at E:T (Effector:Target ratio) of 0.5:1, 1:1, and 2:1, as compared to HA-CD16 KHYG1 alone. Furthermore, the combination was also significantly cytotoxic against NK sensitive cell line RPMI 8226, albeit at a lower NK:MM E:T ratio E:T 0.25:1, 1:1.

### Example 5

### Demonstration that Daratumumab Induces Minimal Collateral Damage on CD16^{+ KHYG}1 NK Cells.

CD16 expressing KHYG1 cells were co-cultured with multiple myeloma cell lines RPMI 8226, H929, JJN3 and U266 or primary CD38⁺ multiple myeloma cells either alone or in combination with Daramutumab in an ADCC assay. NK cell induced cytotoxicity was measured by FACS-based methods as described below. The results for multiple myeloma cell lines RMPI 8226, H929, JJN3 and U266 are shown in FIGS. 6A-6B, which demonstrate that the presence of Daramutumab in the assay had no significant effect on the viability of the CD16 expressing KHYG1 NK cells. The results for the primary CD38⁺ multiple myeloma cells are shown in FIGS. 10A-10D which also demonstrate that the presence of Daramutumab in the assay had no significant effect on the viability of the CD16 expressing KHYG1 NK cells.

### Example 6

### The Combination of Daratumumab and CD16⁺ KHYG1 Eliminates Cells of Multiple Myeloma Cell Lines

CD16 expressing KHYG1 cells were co-cultured with multiple myeloma cell lines RMPI 8226, H929, JJN3 and U266 or primary CD38⁺ multiple myeloma cells either alone or in combination with Daramutumab in an ADCC assay at E:T (Effector:Target) ratios of 0:1, 0.25:1, 0.5:1, 1:1 and 2:1. FIGS. 7A-7B show the results for CD38^{high} multiple myeloma cell lines RPMI-8266 and H929. FIGS. 8A-8B show the results for CD38^{low} multiple myeloma cell lines JJN3 and U266. FIGS. 8A-8B show the results for primary CD38⁺ multiple myeloma cells. All of these experiments demonstrate that CD16 expressing KHYG1 cells are cytotoxic to the multiple myeloma cell lines and primary multiple myeloma cells tested.

### Example 7

Demonstration of CD16 receptor shedding upon interaction with multiple myeloma cells CD16 expressing KHYG1 cells were co-cultured for 24 hours with multiple myeloma cell line RMPI 8226, H929, JJN3 and U266 or primary CD38⁺ multiple myeloma cells either alone or in combination with Daramutumab in an ADCC assay. FIGS. 10A-10D show the results of a 24-hour ADCC assay with CD16 mRNA nucleofected KHYG1 against H929 cells with or without Daratumumab. FIGS. 10A-10D show that CD16⁺ KHYG1 NK in combination with Daratumumab exhibits very limited shedding of the CD16 receptor upon interaction (i.e. activation) with cells of the H929 (multiple myeloma) cell line.

### Example 8

### Demonstration that CD16 Expressing KHYG1 Induces Cell Death in Daratumumab Treated Primary Multiple Myeloma Cells

CD16 expressing KHYG1 cells were analyzed for surface expression of CD16, and further co-cultured with primary multiple myeloma cells from 5 different multiple myeloma patients in combination with Daramutumab in an ADCC assay. As control, primary myeloma cells were cultured with mock nucleofected KHYG1 in the presence of Daratumumab at different E:T (Effector:Target) ratios.

CD16 nucleofected KHYG1 in combination with Daramutumab was significantly more cytotoxic towards the primary multiple myeloma cells (FIGS. 9A-9E; data represents mean of 5 independent experiments at E:T ratios of 0.5:1, 1:1, 2.5:1 and 5:1) as compared to mock nucleofected KHYG1 and Daratumumab.

### Example 9

Demonstration of cytokine release during a 14-hour ADCC measured by ELISA for interferon gamma (IFN-γ) (FIGS. 12A-12B); and TNF-α (FIGS. 12C-12D) in CD16 expressing KHYG1 in combination with Daratumumab when co-cultured with multiple myeloma cell lines H929 and JJN3. Standard curves for the ELISA assays were carried out and it was confirmed that the quantities measured for the experiments presented fell within the linear range of the ELISA.

### Example 10

Breast cancer cell lines HCC-1954, MDA-MB-453 and ZR-75-1 were shown (via FACS) to express HER2 at differing levels (see FIGS. 13A, 13B, 13C and 13D). HCC-1954 was shown to express HER2 to a further extent than either of the other two cell lines.

In FIGS. 14A-14C, it is shown that at different E:T ratios KHYG1 cells transfected to transiently express CD16 mRNA in combination with Herceptin are more effective at killing the 3 cancer cell lines than mock KHYG1 cells with Herceptin. This specifically shows that ADCC is enhanced when the KHYG1 cells are transfected with CD16 mRNA.

Finally, it is shown in FIGS. 15A-15C that the addition of immunosuppressive factors lactate (50 mM), PGE₂ (100 ng/mL) and TGF-β (5 ng/mL) individually or in combination was not effective at reducing the enhanced ADCC demonstrated against the 3 breast cancer cell lines by the KHYG1 cells transfected with CD16 mRNA. This provides evidence that expression of CD16 in NK cells, according to the disclosure, is effective at mitigating immunosuppression.

### Materials and Methods

### Electroporation of CD16 mRNA into KHYG1 NK cells

### Electroporation

One Sample Contains

100 μl (OC-100 cuvette Maxcyte GT)

Cell number: 2×10⁶ cells

Maxcyte Buffer: 100 μl (for each sample)

CD16 mRNA 12.5 ug/100 μl sample
1. Passage cells at 1:1 (10 ml cells+10 ml media) on the day before electroporation in T75 flask, cells must be in logarithmic growth phase.
2. Pre-warm the Maxcyte Buffer to room temperature.
3. Prepare a fresh 10 ml aliquot of culture medium (CM) containing 2 ml FBS, 8 ml RPMI 1640, and supplements 1 μl IL-2 (RPMI1640+20% FBS+100 IU/ml IL-2) at 37° C. in a 15 ml tube (no antibiotics).
4. Take 10 ml cell culture in 15 ml tubes and count the cells to determine the cell density.
5. Spin cells at 1200 rpm/5 min and discard the supernatant.
6. Wash cells once with 5 ml Maxcyte Buffer.
7. Resuspend 2×10⁶ in 100 μl Maxcyte Buffer.
8. Transfer the sample into an OC-100 cuvette. Add 12.5 ug mRNA 12.5 ug/100 μl sample to the “CD16+ KHYG1” cuvette. The “MOCK KHYG1” cuvette will not contain any mRNA. Make sure that the sample covers the bottom of the cuvette, avoid air bubbles while pipetting.
9. Close cuvette with the cap.
10. Select a program for natural killer cells on the Maxcyte GT. Insert the “MOCK KHYG1” cuvette into the cuvette holder press the start program. Repeat this for “CD16+ KHYG1” cuvette.
11. Remove the cuvette and transfer the cells as a “bubble” to a 6 well plate. Use two separate wells, one for “CD16+ KHYG1” and another for “MOCK KHYG1”
12. Incubate cells in a humidified 37° C. for 20 minutes. After 20 minutes add 3 ml of CM to each well.
13. Incubate cells in a humidified 37° C. incubator for another 24 hours.
14. Measure CD16 expression on the “MOCK KHYG1” and “CD16+ KHYG1”
15. Set up cytotoxicity assay as described below with MM cell lines.

### Time Course Experiments

16. Incubate cells suspension for up to 120 hours at 37° C. & 5% CO₂ post electroporation. Add 1-4 ml fresh media (RPMI1640+10% FBS+100 IU/ml IL-2) as per cell growth requirements.

Note: The cell culture is examined every 24 hours under a microscope to check the status and condition of the cells.

### Nucleofection of CD16 mRNA into KHYG1 NK Cells

### Nucleofection

One Nucleofection sample contains:

100 μl (standard cuvette)

Cell number: 2×10⁶ cells

Nucleofector solution: 100 μl (Supplement 18 μl+freshly prepared Nucleofector solution

T, 82 μl, (Incubate at 37° C. incubator for 10 minutes) (15 ml tubes))

Amaxa Nucleofector 11 system
1. Pre-warm the solution T to room temperature.
2. Prepare a fresh 10 ml aliquot of culture medium containing 2 ml FBS, 8 ml RPMI 1640, and supplements 1 μl IL-2 (IL-2 10 ng/ml) at 37° C. in a 15 ml tube (no antibiotics!!!).
3. Prepare 12-well plates by filling with 2 ml of culture medium containing the above media, and pre-incubate plates in a humidified 37° C. incubator for 20 minutes.
4. Take 10 ml cell culture in 15 ml tubes and count the cells to determine the cell density.
5. Centrifuge the required number of cells 2×10⁶ at 1200 rpm for 5 min.
6. Discard supernatant completely so that no residual medium covers the cell pellet.
7. Resuspend the cell pellet in room temperature 100 μl Nucleofector Solution (see above) to a final concentration of 2×10⁶ cells/100 μl.
8. Avoid storing the cell suspension longer than 5 min in Nucleofector Solution, as this reduces cell viability and gene transfer efficiency.
9. Add 12.5 ug m-RNA one tube.
10. Transfer the sample into an Amaxa certified cuvette.
11. Make sure that the sample covers the bottom of the cuvette, avoid air bubbles while pipetting.
12. Close cuvette with the blue cap.
13. Select Nucleofector program (A-024). Insert the cuvette into the cuvette holder press the “X” button to start the program.
14. To avoid damage to the cells, remove the samples from the cuvette immediately after the program has finished (display showing “OK”).
15. Add the pre-warmed culture medium into the cuvette and transfer the sample into the prepared 12-well plated.
16. Press the “X” button to reset the Nucleofector.
17. Incubate cells in a humidified 37° C. incubator for 24 hours.
18. Perform flow cytometric analysis at 24 hour time point.
19. Set up cytotoxicity assay as described below with MM cell lines

### Cell Cytotoxicity Assay (Part 1 with Maxcyte GT against MM Cell Lines

1. Count MM cell lines RPM1-8226, H929, JJN3, and U266.
2. Incubate 400,000 MM cells in 1 ml RPMI1640 media (+10% FBS+1%P/S) with Daratumumab at a final concentration of 10 ug/ml.
3. Incubate the cells at room temperature for 30 minutes with Daratumumab.
4. Plate 100 μl (40,000 cells) of Daratumumab treated MM cells in 96 well plate, either alone, or in-combination with NK cells—“MOCK KHYG1” or “CD16+ KHYG1” as below.
5. Add 100 μl of “MOCK KHYG1” or “CD16+ KHYG1” NK cells containing 40,000 cells to the MM cells
6. Incubate the co-cultures at 37° C. incubator for 14 hours. Perform “Cell staining Protocol for cytotoxicity”

### Cell Cytotoxicity Assay (Part 2 with Amaxa Nucleofector II against MM Cell Lines)

1. Count MM cell lines RPMI-8226, H929, JJN3, and U266.
2. Incubate 400,000 MM cells in 1 ml RPMI1640 media (+10% FBS+1% P/S) with or without Daratumumab at a final concentration of 10 ug/ml.
3. Incubate the cells at room temperature for 30 minutes with Daratumumab.
4. Plate 100 μl (40,000 cells) of untreated MM cells “or” Daratumumab treated MM cells in 96 well plate, either alone or in-combination with “CD16+ KHYG1” at multiple E:T ratios.
5. Add 100 μl of “CD16+KHYG1” NK cells to the MM cells at the E:T ratio of 0.25:1, 0.5:1, 1:1 and 2:1.
6. Incubate the co-cultures at 37° C. incubator for 14 hours.
7. Perform “Cell staining Protocol for cytotoxicity”

### Cell Cytotoxicity Assay (Part 3 with Maxcyte GT against Primary Patient Derived CD38⁺ Cells

1. Isolate CD38⁺ MM cells from the patient Bone marrow. Check the expression of CD38 on the isolated cells.
2. Count the CD38⁺ MM cells.
3. Incubate 100,000 MM cells in 0.5 ml RPMI1640 media (+10% FBS+1% P/S) with Daratumumab at a final concentration of 10 ug/ml.
4. Incubate the cells at room temperature for 30 minutes with Daratumumab.
5. Plate 100 μl (20,000) of Daratumumab treated MM cells in 96 well plate, either alone, or in-combination with NK cells—“MOCK KHYG1” or “CD16+ KHYG1” as below.
6. Add 100 μl of “MOCK KHYG1” or “CD16+ KHYG1” NK cells at the E:T ratio of 0.25:1, 0.5:1, 1:1 and 2:1.
7. Incubate the co-cultures at 37° C. incubator for 14 hours. Perform “Cell staining Protocol for cytotoxicity”

### Cell Staining Protocol for Cytotoxicity

1. Prefill FACS tubes with 200 μl FACS buffer (15 tubes) with Eppendorf repeater unit.
2. Add the cell co-cultures to the FACS tubes.
3. Spin at 2000 RPM/3 MIN
4. Discard supernatant by inverting on a try and then botting on a dry paper.
5. Resuspend cells in the tubes by vortexing.
6. Add 1 μl of diluted CD2 BV421 antibody
7. Incubate for 25 mins on dark/ice.
8. Add 200 μl FACS buffer (30 tubes) with Eppendorf repeater unit
9. Spin at 2000 RPM/3 MIN
10. Discard supernatant by inverting on a try and then botting on a dry paper.
11. Resuspend cells in the tubes by vortexing
12. Add 200μl FACS buffer (30 tubes) to each tube with Eppendorf repeater unit
13. Measure on FACS CANTO II
14. Add 2 μl of propidium iodide in each tube, wait 2-3 mins and measure each tube.

### Staining Protocol for CD38

1. Obtain 1×10⁶ cells NK cells, NK92, KHYG1 and primary expanded NK cells
2. Centrifuge at 2000 rpm for 3 mins.
3. Discard supernatant, add 5 ml FACS buffer and centrifuge at 200 rpm for 3 mins.
4. Resuspend the 1×10⁶ cells in 250 μl in FACS buffer.
5. Aliquot 50 μl of cells and antibody in each tubes as per below:
   Unstained
   CD38-Pe antibody
6. Mix well, vortex for 1-3 seconds, and incubate for 15 minutes in the dark in the refrigerator (2-8 ° C.) (NB this step should not be carried out on ice).
7. Wash cells with 0.5 ml of buffer and centrifuge at 2000 rpm for 3 minutes. Aspirate the supernatant completely.
8. Resuspend cell pellet in a suitable amount of buffer (200 μl) for analysis by FACS.

### Staining Protocol for KIR expression

9. Obtain 1×10⁶ cells NK cells, NK92, KHYG1 and primary expanded NK cells
10. Centrifuge at 2000 rpm for 3 mins.
11. Discard supernatant, add 5 ml FACS buffer and centrifuge at 200 rpm for 3 mins.
12. Resuspend the 1×10⁶ cells in 250 μl in FACS buffer.
13. Aliquot 50 μl of cells and antibody in each tubes as per below:
   i) Unstained
   ii) KIR 2DL1
   iii) KIR2DL2/3
   iv) KIR3DL1
14. Mix well, vortex for 1-3 seconds, and incubate for 15 minutes in the dark in the refrigerator (2-8 ° C.) (NB this step should not be carried out on ice).
15. Wash cells with 0.5 ml of buffer and centrifuge at 2000 rpm for 3 minutes. Aspirate the supernatant completely.
16. Resuspend cell pellet in a suitable amount of buffer (200 μl) for analysis by FACS.

The disclosure thus provides NK cells and cell lines, and production thereof, for use in blood cancer therapy.

1. A natural killer (NK) cell transiently expressing an Fc receptor from an extra-chromosomal nucleic acid.

2. The NK cell of claim 1, wherein the extra-chromosomal nucleic acid is mRNA.

3. The NK cell of claim 1, wherein the Fc receptor is CD16.

4. The NK cell of claim 3, wherein the CD16 receptor comprises the amino acid substitution mutation F158V.

5. The NK cell of claim 1, wherein the NK cell is CD38^{low}.

6. The NK cell of claim 1, wherein the NK cell is of the KHYG-1 cell line.

7. A method of treating cancer in a human patient, comprising administering to the patient an NK cell in combination with an antibody, wherein the NK cell transiently expresses an Fc receptor from an extra-chromosomal nucleic acid.

8. The method of claim 7, wherein the cancer is selected from the group consisting of acute myeloid leukemia, multiple myeloma and breast cancer.

9. The method of claim 7, wherein the antibody is selected from the group consisting of Daratumumab, Trastuzumab, Alemtuzumab, Brentuximab, Blinatumomab, Pankomab, Avelumab, Durvalumab and Atezolizumab.

10. The method of claim 7, wherein the extra-chromosomal nucleic acid is mRNA.

11. The method of claim 7, wherein the Fc receptor is CD16.

12. The method of claim 11, wherein the CD16 receptor comprises the amino acid substitution mutation F158V.

13. The method of claim 7, wherein the NK cell is CD38^{low}.

14. The method of claim 7, wherein the NK cell is of the KHYG-1 cell line.

15. A pharmaceutical kit comprising (a) the NK cell of claim 1; (b) an antibody; and (c) instructions for administration of the NK cell and the antibody to a patient.

16. The pharmaceutical kit of claim 15, wherein the antibody binds CD38.

17. The pharmaceutical kit of claim 15, wherein the antibody binds HER2.

18. The pharmaceutical kit of claim 15, wherein the antibody is selected from the group consisting of Daratumumab, Trastuzumab, Alemtuzumab, Brentuximab, Blinatumomab, Pankomab, Avelumab, Durvalumab and Atezolizumab.

19. A method of treating a CD38-expressing cancer in a human patient, comprising administering to the patient a CD38^{low} NK cell expressing an Fc receptor in combination with a CD38-binding antibody.

20. The method of claim 19, wherein the CD38-binding antibody is Daratumumab.
